SEQUENCE LISTING (21 sequences)

SEQ ID NO: 1 - DNA, 114 bp, Fusarium longipes
  CDS (1)..(111)
cagtcccccatctggggacagtgtggtggaaacggatggactggtgcaacaacatgtcagtccggactcaagtgtgagaaagtgaacgattggtactaccagtgtgtcccctaa

SEQ ID NO: 2 - protein, 37 aa, Fusarium longipes
QSPIWGQCGGNGWTGATTCQSGLKCEKVNDWYYQCVP

SEQ ID NO: 3 - DNA, 144 bp, Fusarium longipes
  CDS (1)..(141)
gcaccggtcgaagaacgacagtcgtgttcgaacggagtctgggcacagtgtggtggtcagaactggtcgggtacaccctgttgtacatccggcaacacatgtgtcaaaatcaacgacttctactcgcagtgtcagcctggctaa

SEQ ID NO: 4 - protein, 47 aa, Fusarium longipes
APVEERQSCSNGVWAQCGGQNWSGTPCCTSGNTCVKINDFYSQCQPG

SEQ ID NO: 5 - DNA, 123 bp, Aspergillus clavatus
  CDS (1)..(120)
cagcagtccctctatggccagtgtggaggtaacggctggtccggacccacagagtgtacagcaggagcatgttgtcaggtccagaacccgtggtattcccagtgtctccctggcgattgttaa

SEQ ID NO: 6 - protein, 40 aa, Aspergillus clavatus
QQSLYGQCGGNGWSGPTECTAGACCQVQNPWYSQCLPGDC

SEQ ID NO: 7 - DNA, 381 bp, Ruminococcus flavefaciens
  CDS (1)..(381)
gatggttacaccattaagcccaacaagaaagtcacttactcggcactcggcgaagatgaacggatgattggcttctcgtacaaggacttcggcatctcctcgtcggaaaagatcacagaggtccaggtcaacatttcggccaacaagaacattggtaagtacgtcggccagttcggcacgtccacaaccgactcggcaaacggatactgggccatgggcgacgagatcactcagtccatctcgggtaactccggcacgatcacatggaaggtcccctcggatatctcgtcgatcatccagacgcagtatggcggagaaatcaaattcggagtgtggtggatcgattgtgatgagttcacaatcgattcggtggtcctcaaa

SEQ ID NO: 8 - protein, 127 aa, Ruminococcus flavefaciens
DGYTIKPNKKVTYSALGEDERMIGFSYKDFGISSSEKITEVQVNISANKNIGKYVGQFGTSTTDSANGYWAMGDEITQSISGNSGTITWKVPSDISSIIQTQYGGEIKFGVWWIDCDEFTIDSVVLK

SEQ ID NO: 9 - DNA, 567 bp, Artificial Sequence
  Unidentified microorganism
  CDS (1)..(567)
ggctacaagtacccgacagccgacgatttcgaaatcgtgtatgacatctcgtacaacgacgagtggtccgaattgttcttgttcggctcgtgggacaggactgccgtcaacttgtcgggatacaagggcatccgcgtggagatggacaaggcctatggcaacaaactccagatcaaggtgtacggcgacaagaagtccggtaccgatttcaacgaacagtatgcccctctctccgatacatcggcctccacgacggtcgatttcgacacctcgattttgggctcgacgttctggggtgtcacgttgcagacgaactccggtgcattgaccgcgacactcaaagaggccaagttgatcaaggccgacggaaccgaggaacctgcctcggtgaccgcagcatggggatgtacagtgactgccaagtcgaccccgaaacctaccggcatccacgccatccagttgatcaaaaccgaagcagatggtgccatctataacctccagggccagagggtgcagaacccccagaagggtatctacattcagaacggcaagaaatacgtgatgaaa

SEQ ID NO: 10 - protein, 189 aa, Artificial Sequence
  Synthetic Construct
GYKYPTADDFEIVYDISYNDEWSELFLFGSWDRTAVNLSGYKGIRVEMDKAYGNKLQIKVYGDKKSGTDFNEQYAPLSDTSASTTVDFDTSILGSTFWGVTLQTNSGALTATLKEAKLIKADGTEEPASVTAAWGCTVTAKSTPKPTGIHAIQLIKTEADGAIYNLQGQRVQNPQKGIYIQNGKKYVMK

SEQ ID NO: 11 - DNA, 435 bp, Hungateiclostridium thermocellum
  CDS (1)..(435)
ggcactctcggaggattcactacctccggcaccaacgccacaggagtggtggtgaacacgaccgagaaggcattcaagggagagaggggactcaagtggacagtcacatcggagggtgagggtactgccgagttgaagttggatggaggcacaatcgtcgtccctggcacgactatgactttccggatctggattccctcgggtgcacctattgcagccatccagccttacatcatgcctcatacaccggattggtcggaggtcctctggaactcgacctggaagggatacacgatggtcaagacggatgattggaacgagattaccctcactctcccggaagacgtggaccccacttggcctcagcagatgggaattcaggtccagaccatcgacgaaggcgaattcacaatctacgtggatgcgatcgattgg

SEQ ID NO: 12 - protein, 145 aa, Hungateiclostridium thermocellum
GTLGGFTTSGTNATGVVVNTTEKAFKGERGLKWTVTSEGEGTAELKLDGGTIVVPGTTMTFRIWIPSGAPIAAIQPYIMPHTPDWSEVLWNSTWKGYTMVKTDDWNEITLTLPEDVDPTWPQQMGIQVQTIDEGEFTIYVDAIDW

SEQ ID NO: 13 - DNA, 552 bp, Clostridium cellulovorans
  CDS (1)..(552)
gataccacagtgtccaggaagctcatggatctcgaggtgttcaagtccgcatccattaccggctggtccggatcggcaggaggcgaactcgaggtggcatccgattcgaacttgcccatcgacacatccgcaacctacaacggcttgccttccttgaggttgaacgtcacaaaggcctccgcacagtggtggtcctcgctcctcacactcaggggatggtgtactcaggacttgacacagtacctcgccaacggctatttggagttcaacgtgaaaggtaaggtcggaggcgaggacttccagattggactccaggaccagactcatgaacgagcagccggagactcggtcacctccgtgaagtcgatcaagaactatgtcaacatctcgaccaactggcagcacgtgaagatccccttgaaggacattatgggaccctccactggattcgacccgactacagcccgatgtatcaacatcgtgaagggctcctcggagatcttcacggcatggatcaacgacctcaagatcacgtcgacggacaacgagaag

SEQ ID NO: 14 - protein, 184 aa, Clostridium cellulovorans
DTTVSRKLMDLEVFKSASITGWSGSAGGELEVASDSNLPIDTSATYNGLPSLRLNVTKASAQWWSSLLTLRGWCTQDLTQYLANGYLEFNVKGKVGGEDFQIGLQDQTHERAAGDSVTSVKSIKNYVNISTNWQHVKIPLKDIMGPSTGFDPTTARCINIVKGSSEIFTAWINDLKITSTDNEK

SEQ ID NO: 15 - DNA, 1083 bp, Clostridium cellulovorans
  CDS (1)..(1083)
ttgtgggatttcaacgacggcaccaagcagggcttcggcgtcaacggcgattcccctgtcgaagatgtggtcatcgagaacgaggcaggtgccttgaagctctccggcttggatgcctccaacgacgtctcggagggaaactactgggcaaacgcgaggctctcggcagatggatggggcaaatcggtggacattttgggtgcagagaaactcaccatggatgtcatcgtggacgaaccgacgaccgtctccattgcagccatccctcagggtccttccgcgaactgggtgaaccccaaccgtgccattaaggtcgagcccaccaacttcgtgcccttgggcgataagttcaaggcggaactcacaatcacgtccgcagactcgccttccctcgaagcaattgccatgcacgcagagaacaacaacatcaacaacatcatcctcttcgtgggaacggaaggtgccgacgtcatttacctcgataacatcaaagtgatcggtacagaagtggaaattcccgtggtccacgatcccaagggcgaggccgtgctcccctcggtcttcgaagatggtaccaggcagggatgggattgggcaggtgagtcgggtgtgaagactgccctcacaatcgaggaagccaacggatcgaacgcgctctcctgggaattcggctaccccgaagtcaaaccgtcggacaactgggcaacagcaccgaggctcgacttctggaagtcggacttggtgaggggagagaacgactacgtgactttcgacttctatctcgatcctgtgagggcaaccgaaggcgcaatgaacattaacctcgtcttccagcctcccacgaacggatactgggtgcaggcacctaaaacttacaccatcaacttcgatgagctcgaagaggccaaccaggtgaacggcttgtatcactacgaggtcaagattaacgtccgcgatatcacaaacatccaggacgacacactcttgaggaacatgatgatcatcttcgccgacgtcgaatcggatttcgcaggacgcgtcttcgtggacaacgtccggttcgagggagcagcgaccactgag

SEQ ID NO: 16 - protein, 361 aa, Clostridium cellulovorans
LWDFNDGTKQGFGVNGDSPVEDVVIENEAGALKLSGLDASNDVSEGNYWANARLSADGWGKSVDILGAEKLTMDVIVDEPTTVSIAAIPQGPSANWVNPNRAIKVEPTNFVPLGDKFKAELTITSADSPSLEAIAMHAENNNINNIILFVGTEGADVIYLDNIKVIGTEVEIPVVHDPKGEAVLPSVFEDGTRQGWDWAGESGVKTALTIEEANGSNALSWEFGYPEVKPSDNWATAPRLDFWKSDLVRGENDYVTFDFYLDPVRATEGAMNINLVFQPPTNGYWVQAPKTYTINFDELEEANQVNGLYHYEVKINVRDITNIQDDTLLRNMMIIFADVESDFAGRVFVDNVRFEGAATTE

SEQ ID NO: 17 - protein, 166 aa, Clostridium cellulovorans
LWDFNDGTKQGFGVNGDSPVEDVVIENEAGALKLSGLDASNDVSEGNYWANARLSADGWGKSVDILGAEKLTMDVIVDEPTTVSIAAIPQGPSANWVNPNRAIKVEPTNFVPLGDKFKAELTITSADSPSLEAIAMHAENNNINNIILFVGTEGADVIYLDNIKVI

SEQ ID NO: 18 - protein, 195 aa, Clostridum cellulovorans
GTEVEIPVVHDPKGEAVLPSVFEDGTRQGWDWAGESGVKTALTIEEANGSNALSWEFGYPEVKPSDNWATAPRLDFWKSDLVRGENDYVTFDFYLDPVRATEGAMNINLVFQPPTNGYWVQAPKTYTINFDELEEANQVNGLYHYEVKINVRDITNIQDDTLLRNMMIIFADVESDFAGRVFVDNVRFEGAATTE

SEQ ID NO: 19 - DNA, 456 bp, Cellulomonas fimi
  CDS (1)..(456)
gcatcgccgattggtgagggtaccttcgatgatggtcccgagggctgggtcgcatatggtaccgatggtcccttggatacgtcgacaggagcactctgtgtcgcagtgcctgcaggttccgcacagtacggtgtcggagtggtcttgaacggagtcgcaatcgaggagggtaccacgtatacgctccgttatactgcaaccgcatccacggatgtcacagtccgagcactcgtgggacagaacggagcaccctatggaactgtcctcgatacatcgcctgcactcacatccgaacctcgacaggtcaccgaaaccttcacagcatcggcaacttatcctgcaacgcctgcagcagatgatcctgagggtcagatcgcattccagttgggaggcttctcggcagatgcatggaccttctgtttggatgatgtcgcattggattcggaggtcgagttg

SEQ ID NO: 20 - protein, 152 aa, Cellulomonas fimi
ASPIGEGTFDDGPEGWVAYGTDGPLDTSTGALCVAVPAGSAQYGVGVVLNGVAIEEGTTYTLRYTATASTDVTVRALVGQNGAPYGTVLDTSPALTSEPRQVTETFTASATYPATPAADDPEGQIAFQLGGFSADAWTFCLDDVALDSEVEL

SEQ ID NO: 21 - protein, 19 aa, Artificial Sequence
  Signal peptide sequence
MKLSWLVAAALTAASVVSA

(19) United States
(12) Patent Application Publication
(10) Pub. No.: US 2021/0009927 A1
(43) Pub. Date: Jan. 14, 2021

(54) Polypeptides Comprising Carbohydrate Binding Activity in Detergent Compositions And Their use in Reducing Wrinkles in Textile or Fabrics

(71) Applicant: Novozymes A/S, Bagsvaerd (DK)

(72) Inventors: Lone Baunsgaard, Helsingor (DK); Kenneth Jensen, Oelsted (DK); Marc Dominique Morant, Frederiksberg (DK)

(73) Assignee: Novozymes A/S, Bagsvaerd (DK)

(21) Appl. No.: 17/040,710

(22) PCT Filed: Apr. 12, 2019
(86) PCT No.: PCT/EP2019/059510

§ 371 Date: Sep. 23, 2020

(30) Foreign Application Priority Data
Apr. 17, 2018 (EP) 18167742.8

(51) Int. Cl. C11D 3/386; C11D 11/00

(52) U.S. Cl. CPC C11D 3/38681; C11D 11/0017

(57) ABSTRACT

Disclosed is the use of polypeptides having carbohydrate binding properties, such as CBM, for reducing the wrinkles in laundry. Also detergent compositions comprising CBM are disclosed.

### REFERENCE TO SEQUENCE LISTING

This application contains a Sequence Listing in computer readable form. The computer readable form is incorporated herein by reference.

### FIELD OF THE INVENTION

The present invention relates to detergents for laundry. In particular the invention relates to the use of carbohydrate binding modules to provide an anti-wrinkle effect to textile.

### BACKGROUND OF THE INVENTION

Laundering of textiles is common activities in normal household activities. When clothes have been used it is typically laundered in order to remove dirt and refresh the clothes before it is used again. Most used laundry processes involved washing in an aqueous detergent solution followed by one or more rinses and subsequent drying.

However, it is also commonly experienced that clothes and textiles becomes wrinkled during laundry, and the washed clothes get a wrinkled, less appealing appearance.

It is desirable to reduce the amount of wrinkles formed during laundry of clothes or textiles.

### SUMMARY OF THE INVENTION

The invention relates to the use of a polypeptide having carbohydrate binding activity for reducing wrinkles and/or providing increased anti-crease properties and/or providing improved ease of ironing and/or providing improved shape retention in a cleaning process of a fabric or textile.

The polypeptide having carbohydrate binding activity is preferably selected among polypeptides known as Carbohydrate binding Modules (CBM) or mixtures thereof.

The invention also relates to a detergent compositions, as well as laundry booster compositions comprising a polypeptide having carbohydrate binding activity.

### Definitions

As used herein, the singular forms “a”, “an”, and “the” are intended to include the plural forms as well, unless the context clearly indicates otherwise.

Anti-wrinkle and anti-crease and reducing wrinkle and wrinkle reduction: In the context of the present invention, the terms “crease” and “wrinkle” and related terms, such as “anti-crease,” “anti-wrinkle,” “reducing wrinkle,” and “wrinkle reduction” refer to non-permanent deformations in fabrics, such as fabrics and textiles which can be removed by flattening at elevated temperature and moisture (e.g. by ironing). The terms are used interchangeably herein.

Bacterial: In the context of the present invention, the term “bacterial” in relation to poly-peptide or carbohydrate binding module refers to a polypeptide encoded by and thus directly derivable from the genome of a bacteria, where such bacteria has not been genetically modified to encode said polypeptide, e.g. by introducing the encoding sequence in the genome by recombinant DNA technology. In the context of the present invention, the term “bacterial carbohydrate binding module” or “carbohydrate binding module obtained from a bacterial source” or “polypeptide is of bacterial origin” thus refers to a polypeptide encoded by and thus directly derivable from the genome of a bacterial species, where the bacterial species has not been subjected to a genetic modification introducing recombinant DNA encoding said polypeptide. Thus, the nucleotide sequence encoding the bacterial polypeptide is a sequence naturally in the genetic background of a bacterial species. A sequence encoding a bacterial polypeptide may also be referred to a wildtype (or parent). The bacterial polypeptide e.g. bacterial carbohydrate binding module also includes naturally occurring polypeptides modified by, e.g., truncation to obtain the portion of the molecule of interest. A bacterial polypeptide includes recombinant produced wild types, as well as synthetically produced peptides. In a further aspect, the invention provides polypeptides substantially homologous to a bacterial polypeptide. In the context of the present invention, the term “substantially homologous” denotes a polypeptide having carbohydrate binding activity which is at least 80%, preferably at least 85%, more preferably at least 90%, more preferably at least 95%, even more preferably at least 96%, 97%, 98%, and most preferably at least 99% identical to the amino acid sequence of a selected bacterial polypeptide.

Carbohydrate binding module: The term “carbohydrate binding module” as used herein refers to the is independent portion of a polypeptide having a contiguous amino acid sequence with a discreet fold and carbohydrate-binding activity. See, e.g., cazy.org/Carbohydrate-Binding-Modules. While CBMs are often naturally occurring within larger enzymes (typically connected via a linker region to one or more catalytic domains), the term as used herein refers to the independent module. A CBM in its naturally occurring form may be located at the N-terminus, C-terminus, or at an internal position of a polypeptide, and as used herein may be a truncation of its naturally occurring form.

Exemplary CBM families useful according to the invention are those of CBM family 1, 4, 17, 28, 30, 44, 72 and 79. Again, with reference to cazy.org/Carbohydrate-Binding-Modules, CBM Family 1 includes modules of approximately 40 residues found almost exclusively in fungi. The cellulose-binding function has been demonstrated in many cases, and appears to be mediated by three aromatic residues separated by about 10.4 angstrom and which form a flat surface. CBM family 4 includes modules of approximately 150 residues found in bacterial enzymes. Binding of these modules has been demonstrated with xylan, beta-1,3-glucan, beta-1,3-1,4-glucan, beta-1,6-glucan and amorphous cellulose but not with crystalline cellulose. CBM family 17 includes modules of approximately 200 residues. Binding to amorphous cellulose, cellooligosaccharides and derivatized cellulose has been demonstrated. Regarding CBM family 28, the module from the endo-1,4-glucanase of Bacillus sp. 1139 binds to non-crystalline cellulose, cellooligosaccharides, and β-(1,3)(1,4)-glucans. For CBM Family 30, binding to cellulose has been demonstrated for the N-terminal module of Fibrobacter succinogenes CeIF. The C-terminal CBM44 module of the Clostridium thermocellum enzyme has been demonstrated to bind equally well cellulose and xyloglucan. CBM Family 72 includes modules of 130-180 residues found at the C-terminus glycoside hydrolases from various families, sometimes as tandem repeats. The CBM72 found on an endoglucanase from an uncultivated microorganism was found to bind a broad spectrum of polysaccharides including soluble and insoluble cellulose, beta-1,3/1,4-mixed linked glucans, xylan, and beta-mannan. CBM Family 79 includes modules of approx. 130 residues found so far only in ruminococcal proteins. Binding to various beta-glucans was shown for the R. flavefaciens GH9 enzyme.

In a preferred embodiment, the carbohydrate binding module is not attached to (linked to) a softening protein.

As used herein “mixture” or “mixtures” of CBM include blends of polypeptides that are otherwise independently identified, as well as naturally occurring or synthetic constructs of polypeptides. For example, the CBMs useful herein may be present in the former of dimers, trimers, tetramers, and other higher order fusion products, either homologous or heterologous, which may optionally further comprise one or more amino acid linker sequences joining the one or more CBMs.

Detergent components: the term “detergent components” is defined herein to mean the types of chemicals which can be used in detergent compositions. Examples of detergent components are alkalis, surfactants, hydrotropes, builders, co-builders, chelators or chelating agents, bleaching system or bleach components, polymers, fabric hueing agents, fabric conditioners, foam boosters, suds suppressors, dispersants, dye transfer inhibitors, fluorescent whitening agents, perfume, optical brighteners, bactericides, fungicides, soil suspending agents, soil release polymers, anti-redeposition agents, enzyme inhibitors or stabilizers, enzyme activators, antioxidants and solubilizers.

Detergent Composition: the term “detergent composition” refers to compositions that find use in the removal of undesired compounds from items to be cleaned, such as textiles. The detergent composition may be used to e.g. clean textiles for both household cleaning and industrial cleaning. The terms encompass any materials/compounds selected for the particular type of cleaning composition desired and the form of the product (e.g., liquid, gel, powder, granulate, paste, or spray compositions) and includes, but is not limited to, detergent compositions (e.g., liquid and/or solid laundry detergents and fine fabric detergents; fabric fresheners; fabric softeners; and textile and laundry pre-spotters/pretreatment). In addition to containing the enzyme of the invention, the detergent formulation may contain one or more additional enzymes (such as proteases, amylases, lipases, cutinases, cellulases, endoglucanases, xyloglucanases, pectinases, pectin lyases, xanthanases, peroxidases, haloperoxygenases, catalases, nucleases and mannanases, or any mixture thereof), and/or detergent adjunct ingredients such as surfactants, builders, chelators or chelating agents, bleach system or bleach components, polymers, fabric conditioners, foam boosters, suds suppressors, dyes, perfume, tannish inhibitors, optical brighteners, bactericides, fungicides, soil suspending agents, anti-corrosion agents, enzyme inhibitors or stabilizers, enzyme activators, transferase(s), hydrolytic enzymes, oxido reductases, bluing agents and fluorescent dyes, antioxidants, and solubilizers.

Fabric improvement: The term “fabric improvement” or “textile improvement” means a benefit not directly related to catalytic stain removal or prevention of re-deposition of soils. Examples of such benefits are anti-backstaining, anti-pilling, anti-shrinkage, anti-wear, anti-wrinkle, improved color appearance, fabric softness, improved shape retention, flame or chemical resistance, anti-odor, anti-UV, water-repellency, anti-microbial, improved association between non-cellulosic and cellulosic textiles, improved static control, improved hand or texture, resistance to chemical, biological, radiological or physical hazard, and/or improved tensile strength. Prevention or reduction of dye transfer from one textile to another textile or another part of the same textile is termed anti-backstaining (also termed dye transfer inhibition). Removal of protruding or broken fibers from a textile surface to decrease pilling tendencies or remove already existing pills or fuzz is termed anti-pilling. Coating or reincorporation or smoothing of protruding or broken fibers is also termed anti-pilling. Prevention of or reduction of a decrease in dimensional size is termed anti-shrinkage. Prevention of or repair of abrasion is termed anti-wear. Prevention of wrinkles, recovery of textile from wrinkling, smoothness of seams, and/or retention of creases after repeated home laundering is termed “anti-wrinkle” or anti-crease. Improvement of the textile-softness or reduction of textile stiffness is termed improved fabric softness. Color clarification of a textile, or enhanced colorfastness to laundering, perspiration, light, chlorine and non-chlorine bleach, heat, or light at high temperature is termed improved color appearance. Resistance to dimensional size change or dimensional size change during home laundering is termed improved shape retention. Elevated combustion temperature or resistance to burning or melting at high temperatures is termed flame resistance. Resistance to chemical reactions, solubilization or degradation in the presence of chemical solvents, acid or alkali is termed chemical resistance. Resistance to adsorption or prevention of the retention of odorous compounds, particularly short chain fatty acids or low vapor pressure organic compounds is termed anti-odor. Opacity to and prevention or repair of oxidative damage caused by UV irradiation is termed anti-UV. Decreased retention of water, or resistance to wetting is termed water repellency. Enhanced microbiostatic or microbiocidal properties are termed antimicrobial. An increase in resistance to induced electrostatic charge of a textile, or increase in decay rate of an induced electrostatic charge in a textile is termed improved static control. Resistance to elongation under force or augmentation of breaking force is termed improved tensile strength.

First-wash: The term “first-wash” means showing improvement or performance benefit effect already during or in the first wash, and is not dependent on one or more subsequent wash step or wash and dry steps in order to achieve the benefit.

Fungal: In the context of the present invention the term “fungal” in relation to polypeptide or carbohydrate binding module refers to a polypeptide encoded by and thus directly derivable from the genome of a fungus, where such fungus has not been genetically modified to encode said polypeptide, e.g. by introducing the encoding sequence in the genome by recombinant DNA technology. In the context of the present invention, the term “fungal carbohydrate binding module” or “carbohydrate binding module obtained from a fungal source” or “polypeptide is of fungal origin” thus refers to a polypeptide encoded by and thus directly derivable from the genome of a fungal species, where the fungal species has not been subjected to a genetic modification introducing recombinant DNA encoding said polypeptide. Thus, the nucleotide sequence encoding the fungal polypeptide may be a sequence naturally in the genetic background of a fungal species. A sequence encoding a fungal polypeptide may also be referred to a wildtype (or parent). The fungal polypeptide e.g. fungal carbohydrate binding module also includes naturally occurring polypeptides modified by, e.g., truncation to obtain the portion of the molecule of interest. A fungal polypeptide includes recombinant produced wild types, as well as synthetically produced peptides. In a further aspect, the invention provides polypeptides substantially homologous to a fungal polypeptide. In the context of the present invention, the term “substantially homologous” denotes a polypeptide having carbohydrate binding activity which is at least 80%, preferably at least 85%, more preferably at least 90%, more preferably at least 95%, even more preferably at least 96%, 97%, 98%, and most preferably at least 99% identical to the amino acid sequence of a selected fungal polypeptide.

Laundering: The term “laundering” relates to both household laundering and industrial laundering and means the process of treating textiles with a solution containing a cleaning or detergent composition of the present invention. The laundering process can for example be carried out using e.g. a household or an industrial washing machine or can be carried out by hand.

Laundry booster: A laundry booster is an additive used to increase the efficacy of a main wash detergent composition.

Sequence identity: The relatedness between two amino acid sequences or between two nucleotide sequences is described by the parameter “sequence identity”. For purposes of the present invention, the sequence identity between two amino acid sequences may be determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the—nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences may be determined using the Needleman-Wunsch algorithm (Needleman and Wunsch, 1970, supra) as implemented in the Needle program of the EMBOSS package (EM-BOSS: The European Molecular Biology Open Software Suite, Rice et al., 2000, supra), prefer-ably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the—nobrief option) is used as the percent identity and is calculated as follows:

(Identical Deoxyribonucleotides×100)/(Length of Alignment−Total Number of Gaps in Alignment).

Textile: The term “textile” means any textile material including yarns, yarn intermediates, fibers, non-woven materials, natural materials, synthetic materials, and any other textile material, fabrics made of these materials and products made from fabrics (e.g., garments and other articles), and is intended to include the term “fabric” as well. The textile or fabric may be in the form of knits, wovens, denims, non-wovens, felts, yarns, and towelling. The textile may be cellulose based such as natural cellulosics, including cotton, flax/linen, jute, ramie, sisal or coir or manmade cellulosics (e.g. originating from wood pulp) including viscose/rayon, cellulose acetate fibers (tricell), lyocell or blends thereof. The textile or fabric may also be non-cellulose based such as natural polyamides including wool, camel, cashmere, mohair, rabbit and silk or synthetic polymers such as nylon, aramid, polyester, acrylic, polypropylene and spandex/elastane, or blends thereof as well as blends of cellulose based and non-cellulose based fibers. Examples of blends are blends of cotton and/or rayon/viscose with one or more companion material such as wool, synthetic fiber (e.g. polyamide fiber, acrylic fiber, polyester fiber, polyvinyl chloride fiber, polyurethane fiber, polyurea fiber, aramid fiber), and/or cellulose-containing fiber (e.g. rayon/viscose, ramie, flax/linen, jute, cellulose acetate fiber, lyocell). Fabric may be conventional washable laundry, for example stained household laundry. When the term fabric or garment is used it is intended to include the broader term textiles as well.

Wash cycle: The term “wash cycle” is defined herein as a washing operation wherein textiles are immersed in the wash liquor, mechanical action of some kind is applied to the textile in order to release stains and to facilitate flow of wash liquor in and out of the textile and finally the superfluous wash liquor is removed. After one or more wash cycles, the textile is generally rinsed and dried.

Wash liquor: The term “wash liquor” is intended to mean the solution or mixture of water and detergents optionally including enzymes used for laundering textiles, for hard surface cleaning or for dishwashing.

### DETAILED DESCRIPTION OF THE INVENTION

The invention relates to the use of polypeptide having carbohydrate binding activity for reducing wrinkles in a cleaning process of a fabric or textile.

Carbohydrate binding activity is in this application intended to mean that the polypeptide in question has the ability to bind to a carbohydrate, in particular to a carbohydrate polymer such as cellulose, hemicellulose or starch. In a preferred embodiment, the CBM is a cellulose binding CBM.

Carbohydrate binding activity is well known in the art and has been described in detail for the carbohydrate binding modules, e.g. in http://www.cazy.org/Carbohydrate-Binding-Modules.html where a Carbohydrate-binding Module family classification is disclosed base on the structure of the polypeptides. This site describes more than 80 CBM families and the family numbering used at this site will also be used in the present application and claims.

In one embodiment, the polypeptide having carbohydrate binding activity is selected among carbohydrate binding modules belonging to the families CBM1; CBM4, CBM17, CBM28, CBM30, CBM44, CBM72 and CBM79

In another embodiment, the polypeptide having carbohydrate binding activity is selected among polypeptides having at least 60% sequence identity to SEQ ID NO: 2, 4, 6, 8, 10, 12, 14, 16, 18, e.g. at least 70%, sequence identity, e.g. at least 80% sequence identity, e.g. at least 90% sequence identity; e.g. at least 95%, sequence identity, e.g. at least 96% sequence identity, e.g. at least 97% sequence identity; e.g. at least 98% sequence identity or at least 99% sequence identity.

In another embodiment, the polypeptide having carbohydrate binding activity is selected among polypeptides having the amino acid sequence of SEQ ID NO: 2, SEQ ID NO: 4, SEQ ID NO:6, SEQ ID NO: 8, SEQ ID NO: 10, SEQ ID NO: 12, SEQ ID NO: 14, SEQ ID NO: 16, SEQ ID NO: 18, or having an amino acid sequence that deviate from one of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, by, 1, 2, 3, 4, 5, 6, 7, 8 or 9 substitutions, insertions or deletions.

In one embodiment of the present invention, the polypeptide having carbohydrate binding activity may according to the present invention be added to a detergent composition in an amount corresponding to 0.001-200 mg of protein, such as 0.005-100 mg of protein, preferably 0.01-50 mg of protein, more preferably 0.05-20 mg of protein, even more preferably 0.1-10 mg of protein per liter of wash liquor.

In one embodiment, the polypeptide having carbohydrate binding activity is joined to another polypeptide used in the laundering process, such as an enzyme. In this embodiment, the amount of polypeptide having carbohydrate binding activity should be calculated based on the weight of the polypeptide having carbohydrate binding activity alone, without the weight of the polypeptide joined thereto.

The CBM, may according to the invention be added during the washing process and in this embodiment, the CBMs are typically incorporated in the detergent composition used for the laundry process. In an alternative embodiment, the CBMs are added during the rinse following the washing process and in this embodiment, the CBMs are typically incorporated in a rinsing aid composition.

In another embodiment, the polypeptide having carbohydrate binding activity is not joined to any other polypeptide.

According to the invention the use of the polypeptide having carbohydrate binding activity can reduce the wrinkles occurring during the laundry process compared with a similar washing process without addition of the polypeptide having carbohydrate activity. The number of wrinkles are according to the invention be assessed using the AATCC (American Association of Textile Chemists and Colorists) test method 124-TM 124 Smoothness Appearance of Fabrics after Home Laundering (https://members.aatcc.org/store/tm124/533/).

According to the invention the score is improved with at least 0.15 units, 0.20 units, 0.25, units, 0.30 units, 0.40 units, preferably at least 0.5 units, preferably at least 0.75 unit, preferably at least 1.0 units, preferably at least 1.25 units, preferably at least 1.5 units, preferably at least 1.75 units, preferably at least 2.0 units or even higher.

According to the invention the fabric improvement can be evaluated by panelist assessment. Panelists are asked to select towel part being the softest and to select T-shirt part being the less creased. After evaluation, distribution is calculated. The softness and anti-crease is indicated with X:Y values, wherein X specifies the % of the panelists preferring real items washed with CBM, and Y specifies the % that prefers real item washed without CBM. The sum of the X and Y values is 100%.

According to the invention, the panelists preferring fabrics washed with CBM vs test panelists preferring fabrics washed without CBM is at least 60:40, preferably at least 70:30, preferably at least 80:20 or preferably at least 90:10. Preferably, the improved softness effect ratio of test panelists preferring fabrics washed with CBM vs test panelists preferring fabrics washed without CBM is at least 60:40, preferably at least 70:30, preferably at least 80:20 or preferably at least 90:10.

The invention is not limited to any particular laundering process but can be applied to any laundering process using laundering equipment as known in the art, such as front loader or top loader washing machines, or even hand wash.

The invention is neither limited by the way the textile is dried after the wash, but the invention can be used in combination with any method for drying the textiles, include line drying or the use of a dryer, such as a tumble dryer.

The invention is not limited to any particular fabric or textile but can be applied to any known textiles such as cotton, PET, rayon, viscose wool and silk and any blends of these. It is however preferred that the textile comprises cellulose.

### Detergent Compositions

In one embodiment, the invention is directed to detergent compositions comprising a polypeptide of the present invention in combination with one or more additional cleaning composition components. The choice of additional components is within the skill of the artisan and includes conventional ingredients, including the exemplary non-limiting components set forth below.

The choice of components may include, for textile care, the consideration of the type of textile to be cleaned, the type and/or degree of soiling, the temperature at which cleaning is to take place, and the formulation of the detergent product. Although components mentioned below are categorized by general header according to a particular functionality, this is not to be construed as a limitation, as a component may comprise additional functionalities as will be appreciated by the skilled artisan.

Surfactants

The detergent composition may comprise one or more surfactants, which may be anionic and/or cationic and/or non-ionic and/or semi-polar and/or zwitterionic, or a mixture thereof. In a particular embodiment, the detergent composition includes a mixture of one or more nonionic surfactants and one or more anionic surfactants. The surfactant(s) is typically present at a level of from about 0.1% to 60% by weight, such as about 1% to about 40%, or about 3% to about 20%, or about 3% to about 10%. The surfactant(s) is chosen based on the desired cleaning application, and may include any conventional surfactant(s) known in the art.

When included therein the detergent will usually contain from about 1% to about 40% by weight of an anionic surfactant, such as from about 5% to about 30%, including from about 5% to about 15%, or from about 15% to about 20%, or from about 20% to about 25% of an anionic surfactant. Non-limiting examples of anionic surfactants include sulfates and sulfonates, in particular, linear alkylbenzenesulfonates (LAS), isomers of LAS, branched alkylbenzenesulfonates (BABS), phenylalkanesulfonates, alpha-olefinsulfonates (AOS), olefin sulfonates, alkene sulfonates, alkane-2,3-diylbis(sulfates), hydroxyalkanesulfonates and disulfonates, alkyl sulfates (AS) such as sodium dodecyl sulfate (SDS), fatty alcohol sulfates (FAS), primary alcohol sulfates (PAS), alcohol ethersulfates (AES or AEOS or FES, also known as alcohol ethoxysulfates or fatty alcohol ether sulfates), secondary alkanesulfonates (SAS), paraffin sulfonates (PS), ester sulfonates, sulfonated fatty acid glycerol esters, alpha-sulfo fatty acid methyl esters (alpha-SFMe or SES) including methyl ester sulfonate (MES), alkyl- or alkenylsuccinic acid, dodecenyl/tetradecenyl succinic acid (DTSA), fatty acid derivatives of amino acids, diesters and monoesters of sulfo-succinic acid or salt of fatty acids (soap), and combinations thereof.

When included therein the detergent will usually contain from about 1% to about 40% by weigh of a cationic surfactant, for example from about 0.5% to about 30%, in particular from about 1% to about 20%, from about 3% to about 10%, such as from about 3% to about 5%, from about 8% to about 12% or from about 10% to about 12%. Non-limiting examples of cationic surfactants include alkyldimethylethanolamine quat (ADMEAQ), cetyltrimethylammonium bromide (CTAB), dimethyldistearylammonium chloride (DSDMAC), and alkylbenzyldimethylammonium, alkyl quaternary ammonium compounds, alkoxylated quaternary ammonium (AQA) compounds, ester quats, and combinations thereof.

When included therein the detergent will usually contain from about 0.2% to about 40% by weight of a nonionic surfactant, for example from about 0.5% to about 30%, in particular, from about 1% to about 20%, from about 3% to about 10%, such as from about 3% to about 5%, from about 8% to about 12%, or from about 10% to about 12%. Non-limiting examples of nonionic surfactants include alcohol ethoxylates (AE or AEO), alcohol propoxylates, propoxylated fatty alcohols (PFA), alkoxylated fatty acid alkyl esters, such as ethoxylated and/or propoxylated fatty acid alkyl esters, alkylphenol ethoxylates (APE), nonylphenol ethoxylates (NPE), alkylpolyglycosides (APG), alkoxylated amines, fatty acid monoethanolamides (FAM), fatty acid diethanolamides (FADA), ethoxylated fatty acid monoethanolamides (EFAM), propoxylated fatty acid monoethanolamides (PFAM), polyhydroxyalkyl fatty acid amides, or N-acyl N-alkyl derivatives of glucosamine (glucamides, GA, or fatty acid glucamides, FAGA), as well as products available under the trade names SPAN and TWEEN, and combinations thereof.

When included therein the detergent will usually contain from about 0.2% to about 10% by weight of a semipolar surfactant. Non-limiting examples of semipolar surfactants include amine oxides (AO) such as alkyldimethylamineoxide, N-(coco alkyl)-N,N-dimethylamine oxide and N-(tallow-alkyl)-N,N-bis(2-hydroxyethyl)amine oxide, and combinations thereof.

When included therein the detergent will usually contain from about 0.2% to about 10% by weight of a zwitterionic surfactant. Non-limiting examples of zwitterionic surfactants include betaines such as alkyldimethylbetaines, sulfobetaines, and combinations thereof.

### Hydrotropes

A hydrotrope is a compound that solubilises hydrophobic compounds in aqueous solutions (or oppositely, polar substances in a non-polar environment). Typically, hydrotropes have both hydrophilic and a hydrophobic character (so-called amphiphilic properties as known from surfactants); however, the molecular structure of hydrotropes generally do not favor spontaneous self-aggregation, see e.g. review by Hodgdon and Kaler (2007), Current Opinion in Colloid & Interface Science 12: 121-128. Hydrotropes do not display a critical concentration above which self-aggregation occurs as found for surfactants and lipids forming miceller, lamellar or other well defined meso-phases. Instead, many hydrotropes show a continuous-type aggregation process where the sizes of aggregates grow as concentration increases. However, many hydrotropes alter the phase behavior, stability, and colloidal properties of systems containing substances of polar and non-polar character, including mixtures of water, oil, surfactants, and polymers. Hydrotropes are classically used across industries from pharma, personal care, food, to technical applications. Use of hydrotropes in detergent compositions allow for example more concentrated formulations of surfactants (as in the process of compacting liquid detergents by removing water) without inducing undesired phenomena such as phase separation or high viscosity.

The detergent may contain 0-10% by weight, for example 0-5% by weight, such as about 0.5 to about 5%, or about 3% to about 5%, of a hydrotrope. Any hydrotrope known in the art for use in detergents may be utilized. Non-limiting examples of hydrotropes include sodium benzenesulfonate, sodium p-toluene sulfonate (STS), sodium xylene sulfonate (SXS), sodium cumene sulfonate (SCS), sodium cymene sulfonate, amine oxides, alcohols and polyglycolethers, sodium hydroxynaphthoate, sodium hydroxynaphthalene sulfonate, sodium ethylhexyl sulfate, and combinations thereof.

### Builders and Co-Builders

The detergent composition may contain about 0-65% by weight, such as about 5% to about 50% of a detergent builder or co-builder, or a mixture thereof. In a dish wash detergent, the level of builder is typically 40-65%, particularly 50-65%. The builder and/or co-builder may particularly be a chelating agent that forms water-soluble complexes with Ca and Mg. Any builder and/or co-builder known in the art for use in laundry detergents may be utilized. Non-limiting examples of builders include zeolites, diphosphates (pyrophosphates), triphosphates such as sodium triphosphate (STP or STPP), carbonates such as sodium carbonate, soluble silicates such as sodium metasilicate, layered silicates (e.g., SKS-6 from Hoechst), ethanolamines such as 2-aminoethan-1-ol (MEA), diethanolamine (DEA, also known as 2,2′-iminodiethan-1-ol), triethanolamine (TEA, also known as 2,2′,2″-nitrilotriethan-1-ol), and (carboxymethyl)inulin (CMI), and combinations thereof. The detergent composition may also contain 0-50% by weight, such as about 5% to about 30%, of a detergent co-builder. The detergent composition may include a co-builder alone, or in combination with a builder, for example a zeolite builder. Non-limiting examples of co-builders include homopolymers of polyacrylates or copolymers thereof, such as poly(acrylic acid) (PAA) or copoly(acrylic acid/maleic acid) (PAA/PMA). Further non-limiting examples include citrate, chelators such as aminocarboxylates, aminopolycarboxylates and phosphonates, and alkyl- or alkenylsuccinic acid. Additional specific examples include 2,2′,2″-nitrilotriacetic acid (NTA), ethylenediaminetetraacetic acid (EDTA), diethylenetriaminepentaacetic acid (DTPA), iminodisuccinic acid (IDS), ethylenediamine-N,N′-disuccinic acid (EDDS), methylglycinediacetic acid (MGDA), glutamic acid-N,N-diacetic acid (GLDA), 1-hydroxyethane-1,1-diphosphonic acid (HEDP), ethylenediaminetetra(methylenephosphonic acid) (EDTMPA), diethylenetriaminepentakis(methylenephosphonic acid) (DTMPA or DTPMPA), N-(2-hydroxyethyl)iminodiacetic acid (EDG), aspartic acid-N-monoacetic acid (ASMA), aspartic acid-N,N-diacetic acid (ASDA), aspartic acid-N-monopropionic acid (ASMP), iminodisuccinic acid (IDA), N-(2-sulfomethyl)-aspartic acid (SMAS), N-(2-sulfoethyl)-aspartic acid (SEAS), N(2-sulfomethyl)-glutamic acid (SMGL), N-(2-sulfoethyl)-glutamic acid (SEGL), N-methyliminodiacetic acid (MIDA), α-alanine-N,N-diacetic acid (α-ALDA), serine-N,N-diacetic acid (SEDA), isoserine-N,N-diacetic acid (ISDA), phenylalanine-N,N-diacetic acid (PHDA), anthranilic acid-N,N-diacetic acid (ANDA), sulfanilic acid-N,N-diacetic acid (SLDA), taurine-N,N-diacetic acid (TUDA) and sulfomethyl-N,N-diacetic acid (SMDA), N-(2-hydroxyethyl)ethylenediamine-N,N,N″-triacetic acid (HEDTA), di-ethanolglycine (DEG), diethylenetriamine penta(methylenephosphonic acid) (DTPMP), aminotris(methylenephosphonic acid) (ATMP), and combinations and salts thereof. Further exemplary builders and/or co-builders are described in, e.g., WO 09/102854, U.S. Pat. No. 5,977,053

### Bleaching Systems

The detergent may contain 0-30% by weight, such as about 1% to about 20%, of a bleaching system. Any bleaching system known in the art for use in laundry detergents may be utilized. Suitable bleaching system components include bleaching catalysts, photobleaches, bleach activators, sources of hydrogen peroxide such as sodium percarbonate, sodium perborates and hydrogen peroxide-urea (1:1), preformed peracids and mixtures thereof. Suitable preformed peracids include, but are not limited to, peroxycarboxylic acids and salts, diperoxydicarboxylic acids, perimidic acids and salts, peroxymonosulfuric acids and salts, for example, Oxone (R), and mixtures thereof. Non-limiting examples of bleaching systems include peroxide-based bleaching systems, which may comprise, for example, an inorganic salt, including alkali metal salts such as sodium salts of perborate (usually mono- or tetra-hydrate), percarbonate, persulfate, perphosphate, persilicate salts, in combination with a peracid-forming bleach activator. The term bleach activator is meant herein as a compound which reacts with hydrogen peroxide to form a peracid via perhydrolysis. The peracid thus formed constitutes the activated bleach. Suitable bleach activators to be used herein include those belonging to the class of esters, amides, imides or anhydrides. Suitable examples are tetraacetylethylenediamine (TAED), sodium 4-[(3,5,5-trimethylhexanoyl)oxy]benzene-1-sulfonate (ISONOBS), 4-(dodecanoyloxy)benzene-1-sulfonate (LOBS), 4-(decanoyloxy)benzene-1-sulfonate, 4-(decanoyloxy)benzoate (DOBS or DOBA), 4-(nonanoyloxy)benzene-1-sulfonate (NOBS), and/or those disclosed in WO98/17767. A particular family of bleach activators of interest was disclosed in EP624154 and particularly preferred in that family is acetyl triethyl citrate (ATC). ATC or a short chain triglyceride like triacetin has the advantage that it is environmentally friendly Furthermore acetyl triethyl citrate and triacetin have good hydrolytical stability in the product upon storage and are efficient bleach activators. Finally ATC is multifunctional, as the citrate released in the perhydrolysis reaction may function as a builder. Alternatively, the bleaching system may comprise peroxyacids of, for example, the amide, imide, or sulfone type. The bleaching system may also comprise peracids such as 6-(phthalimido)peroxyhexanoic acid (PAP). The bleaching system may also include a bleach catalyst. In some embodiments the bleach component may be an organic catalyst selected from the group consisting of organic catalysts having the following formulae:

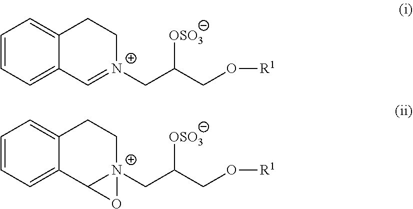

(iii) and mixtures thereof;

wherein each R¹ is independently a branched alkyl group containing from 9 to 24 carbons or linear alkyl group containing from 11 to 24 carbons, preferably each R¹ is independently a branched alkyl group containing from 9 to 18 carbons or linear alkyl group containing from 11 to 18 carbons, more preferably each R¹ is independently selected from the group consisting of 2-propylheptyl, 2-butyloctyl, 2-pentylnonyl, 2-hexyldecyl, dodecyl, tetradecyl, hexadecyl, octadecyl, isononyl, isodecyl, isotridecyl and isopentadecyl. Other exemplary bleaching systems are described, e.g. in WO2007/087258, WO2007/087244, WO2007/087259, EP1867708 (Vitamin K) and WO2007/087242. Suitable photobleaches may for example be sulfonated zinc or aluminium phthalocyanines.

Preferably the bleach component comprises a source of peracid in addition to bleach catalyst, particularly organic bleach catalyst. The source of peracid may be selected from (a) preformed peracid; (b) percarbonate, perborate or persulfate salt (hydrogen peroxide source) preferably in combination with a bleach activator; and (c) perhydrolase enzyme and an ester for forming peracid in situ in the presence of water in a textile or hard surface treatment step.

### Polymers

The detergent may contain 0-10% by weight, such as 0.5-5%, 2-5%, 0.5-2% or 0.2-1% of a polymer. Any polymer known in the art for use in detergents may be utilized. The polymer may function as a co-builder as mentioned above, or may provide antiredeposition, fiber protection, soil release, dye transfer inhibition, grease cleaning and/or anti-foaming properties. Some polymers may have more than one of the above-mentioned properties and/or more than one of the below-mentioned motifs. Exemplary polymers include (carboxymethyl)cellulose (CMC), poly(vinyl alcohol) (PVA), poly(vinylpyrrolidone) (PVP), poly(ethyleneglycol) or poly(ethylene oxide) (PEG), ethoxylated poly(ethyleneimine), carboxymethyl inulin (CMI), and polycarboxylates such as PAA, PAA/PMA, poly-aspartic acid, and lauryl methacrylate/acrylic acid copolymers, hydrophobically modified CMC (HM-CMC) and silicones, copolymers of terephthalic acid and oligomeric glycols, copolymers of poly(ethylene terephthalate) and poly(oxyethene terephthalate) (PET-POET), PVP, poly(vinylimidazole) (PVI), poly(vinylpyridine-N-oxide) (PVPO or PVPNO) and polyvinylpyrrolidonevinylimidazole (PVPVI). Further exemplary polymers include sulfonated polycarboxylates, polyethylene oxide and polypropylene oxide (PEO-PPO) and diquaternium ethoxy sulfate. Other exemplary polymers are disclosed in, e.g., WO 2006/130575. Salts of the above-mentioned polymers are also contemplated.

### Fabric Hueing Agents

The detergent compositions of the present invention may also include fabric hueing agents such as dyes or pigments, which when formulated in detergent compositions can deposit onto a fabric when said fabric is contacted with a wash liquor comprising said detergent compositions and thus altering the tint of said fabric through absorption/reflection of visible light. Fluorescent whitening agents emit at least some visible light. In contrast, fabric hueing agents alter the tint of a surface as they absorb at least a portion of the visible light spectrum. Suitable fabric hueing agents include dyes and dye-clay conjugates, and may also include pigments. Suitable dyes include small molecule dyes and polymeric dyes. Suitable small molecule dyes include small molecule dyes selected from the group consisting of dyes falling into the Colour Index (C.I.) classifications of Direct Blue, Direct Red, Direct Violet, Acid Blue, Acid Red, Acid Violet, Basic Blue, Basic Violet and Basic Red, or mixtures thereof, for example as described in WO2005/03274, WO2005/03275, WO2005/03276 and EP1876226 (hereby incorporated by reference). The detergent composition preferably comprises from about 0.00003 wt % to about 0.2 wt %, from about 0.00008 wt % to about 0.05 wt %, or even from about 0.0001 wt % to about 0.04 wt % fabric hueing agent. The composition may comprise from 0.0001 wt % to 0.2 wt % fabric hueing agent, this may be especially preferred when the composition is in the form of a unit dose pouch. Suitable hueing agents are also disclosed in, e.g. WO 2007/087257 and WO2007/087243.

Enzymes

The detergent additive as well as the detergent composition may comprise one or more enzymes such as a protease, lipase, cutinase, an amylase, carbohydrase, cellulase, pectinase, mannanase, arabinase, galactanase, xylanase, nuclease, oxidase, e.g., a laccase, and/or peroxidase.

In general, the properties of the selected enzyme(s) should be compatible with the selected detergent, (i.e., pH-optimum, compatibility with other enzymatic and non-enzymatic ingredients, etc.), and the enzyme(s) should be present in effective amounts.

Cellulases

Suitable cellulases include those of bacterial or fungal origin. Chemically modified or protein engineered mutants are included. Suitable cellulases include cellulases from the genera Bacillus, Pseudomonas, Humicola, Fusarium, Thielavia, Acremonium, e.g., the fungal cellulases produced from Humicola insolens, Myceliophthora thermophila and Fusarium oxysporum disclosed in U.S. Pat. Nos. 4,435,307, 5,648,263, 5,691,178, 5,776,757 and WO 89/09259.

Especially suitable cellulases are the alkaline or neutral cellulases having colour care benefits. Examples of such cellulases are cellulases described in EP 0 495 257, EP 0 531 372, WO 96/11262, WO 96/29397, WO 98/08940. Other examples are cellulase variants such as those described in WO 94/07998, EP 0 531 315, U.S. Pat. Nos. 5,457,046, 5,686,593, 5,763,254, WO 95/24471, WO 98/12307 and WO99/001544.

Other cellulases are endo-beta-1,4-glucanase enzyme having a sequence of at least 97% identity to the amino acid sequence of position 1 to position 773 of SEQ ID NO:2 of WO 2002/099091 or a family 44 xyloglucanase, which a xyloglucanase enzyme having a sequence of at least 60% identity to positions 40-559 of SEQ ID NO: 2 of WO 2001/062903.

Commercially available cellulases include Celluzyme™, and Carezyme™ (Novozymes NS) Carezyme Premium™ (Novozymes NS), Celluclean™ (Novozymes NS), Celluclean Classic™ (Novozymes NS), Cellusoft™ (Novozymes NS), Whitezyme™ (Novozymes NS), Clazinase™, and Puradax HA™ (Genencor International Inc.), and KAC-500(B)™ (Kao Corporation).

Mannanases

Suitable mannanases include those of bacterial or fungal origin. Chemically or genetically modified mutants are included. The mannanase may be an alkaline mannanase of Family 5 or 26. It may be a wild-type from Bacillus or Humicola, particularly B. agaradhaerens, B. licheniformis, B. halodurans, B. clausii, or H. insolens. Suitable mannanases are described in WO 1999/064619. A commercially available mannanase is Mannaway (Novozymes NS).

### Cellulase

Suitable cellulases include complete cellulases or mono-component endoglucanases of bacterial or fungal origin. Chemically or genetically modified mutants are included. The cellulase may for example be a mono-component or a mixture of mono-component endo-1,4-beta-glucanase often just termed endoglucanases. Suitable cellulases include a fungal cellulase from Humicola insolens (U.S. Pat. No. 4,435,307) or from Trichoderma, e.g. T. reesei or T. viride. Examples of cellulases are described in EP 0 495 257. Other suitable cellulases are from Thielavia e.g. Thielavia terrestris as described in WO 96/29397 or Fusarium oxysporum as described in WO 91/17244 or from Bacillus as described in, WO 02/099091 and JP 2000210081. Other examples are cellulase variants such as those described in WO 94/07998, EP 0 531 315, U.S. Pat. Nos. 5,457,046, 5,686,593, 5,763,254, WO 95/24471, WO 98/12307 Commercially available cellulases include Carezyme®, Celluzyme®, Celluclean®, Celluclast® and Endolase®; Renozyme®; Whitezyme® (Novozymes NS) Puradax®, Puradax HA, and Puradax EG (available from Genencor).

Peroxidases/Oxidases

Suitable peroxidases/oxidases include those of plant, bacterial or fungal origin. Chemically modified or protein engineered mutants are included. Examples of useful peroxidases include peroxidases from Coprinus, e.g., from C. cinereus, and variants thereof as those described in WO 93/24618, WO 95/10602, and WO 98/15257. Commercially available peroxidases include Guardzyme™ (Novozymes NS).

Proteases

Suitable proteases include those of bacterial, fungal, plant, viral or animal origin e.g. vegetable or microbial origin. Microbial origin is preferred. Chemically modified or protein engineered mutants are included. It may be an alkaline protease, such as a serine protease or a metalloprotease. A serine protease may for example be of the 51 family, such as trypsin, or the S8 family such as subtilisin. A metalloproteases protease may for example be a thermolysin from e.g. family M4 or other metalloprotease such as those from M5, M7 or M8 families.

The term “subtilases” refers to a sub-group of serine protease according to Siezen et al., Protein Engng. 4 (1991) 719-737 and Siezen et al. Protein Science 6 (1997) 501-523. Serine proteases are a subgroup of proteases characterized by having a serine in the active site, which forms a covalent adduct with the substrate. The subtilases may be divided into 6 sub-divisions, i.e. the Subtilisin family, the Thermitase family, the Proteinase K family, the Lantibiotic peptidase family, the Kexin family and the Pyrolysin family.

Examples of subtilases are those derived from Bacillus such as Bacillus lentus, Bacillus alkalophilus, Bacillus subtilis, Bacillus amyloliquefaciens, Bacillus pumilus and Bacillus gibsonii described in; U.S. Pat. No. 7,262,042 and WO09/021867, and Subtilisin lentus, Subtilisin Novo, subtilisin Carlsberg, Bacillus licheniformis, subtilisin BPN′, subtilisin 309, subtilisin 147 and subtilisin 168 and e.g. protease PD138 described in (WO93/18140). Other useful proteases may be those described in WO01/016285 and WO02/016547. Examples of trypsin-like proteases are trypsin (e.g. of porcine or bovine origin) and the Fusarium protease described in WO94/25583 and WO05/040372, and the chymotrypsin proteases derived from Cellumonas described in WO05/052161 and WO05/052146.

A further preferred protease is the alkaline protease from Bacillus lentus DSM 5483, as described for example in WO95/23221, and variants thereof which are described in WO92/21760, WO95/23221, EP1921147 and EP1921148.

Examples of metalloproteases are the neutral metalloprotease as described in WO07/044993 (Proctor & Gamble/Genencor Int.) such as those derived from Bacillus amyloliquefaciens.

Examples of useful proteases are the variants described in: WO89/06279 WO92/19729, WO96/034946, WO98/20115, WO98/20116, WO99/011768, WO01/44452, WO03/006602, WO04/03186, WO04/041979, WO07/006305, WO11/036263, WO11/036264, especially the variants with substitutions in one or more of the following positions: 3, 4, 9, 15, 24, 27, 42, 55, 59, 60, 66, 74, 85, 96, 97, 98, 99, 100, 101, 102, 104, 116, 118, 121, 126, 127, 128, 154, 156, 157, 158, 161, 164, 176, 179, 182, 185, 188, 189, 193, 198, 199, 200, 203, 206, 211, 212, 216, 218, 226, 229, 230, 239, 246, 255, 256, 268 and 269 wherein the positions correspond to the positions of the Bacillus lentus protease shown in SEQ ID NO 1 of WO 2016/001449. More preferred the protease variants may comprise one or more of the mutations selected from the group consisting of: S3T, V41, S9R, S9E, A15T, S24G, S24R, K27R, N42R, S55P, G59E, G59D, N60D, N60E, V66A, N74D, S85R, A96S, S97G, S97D, S97A, S97SD, S99E, S99D, S99G, S99M, S99N, S99R, S99H, S101A, V1021, V102Y, V102N, S104A, G116V, G116R, H118D, H118N, A120S, 5126L, P127Q, S128A, 5154D, A156E, G157D, G157P, S158E, Y161A, R164S, Q176E, N179E, S182E, Q185N, A188P, G189E, V193M, N198D, V199I, Y203W, 5206G, L211Q, L211D, N212D, N2125, M2165, A226V, K229L, Q230H, Q239R, N246K, N255W, N255D, N255E, L256E, L256D T268A and R269H. The protease variants are preferably variants of the Bacillus lentus protease (Savinase®) shown in SEQ ID NO 1 of WO2016/001449, the Bacillus amylolichenifaciens protease (BPN′) shown in SEQ ID NO 2 of WO2016/001449. The protease variants preferably have at least 80% sequence identity to SEQ ID NO 1 or SEQ ID NO 2 of WO 2016/001449.

A protease variant comprising a substitution at one or more positions corresponding to positions 171, 173, 175, 179, or 180 of SEQ ID NO: 1 of WO2004/067737, wherein said protease variant has a sequence identity of at least 75% but less than 100% to SEQ ID NO: 1 of WO2004/067737.

Suitable commercially available protease enzymes include those sold under the trade names Alcalase®, Duralase™, Durazym™, Relase®, Relase® Ultra, Savinase®, Savinase® Ultra, Primase®, Polarzyme®, Kannase®, Liquanase®, Liquanase® Ultra, Novozymes Progress®, Novozymes Progress® Uno, Novozymes Progress® Excell, Ovozyme®, Coronase®, Coronase® Ultra, Blaze®, Blaze Evity® 100T, Blaze Evity® 125T, Blaze Evity® 150T, Neutrase®, Everlase® and Esperase® (Novozymes NS), those sold under the tradename Maxatase®, Maxacal®, Maxapem®, Purafect Ox®, Purafect OxP®, Puramax®, FN2®, FN3®, FN4®, Excellase®, Excellenz P1000™, Excellenz P1250™, Eraser®, Preferenz P100™, Purafect Prime®, Preferenz P110™, Effectenz P1000™, Purafect®™, Effectenz P1050™, Purafect Ox®™, Effectenz P2000™, Purafast®, Properase®, Opticlean® and Optimase® (Danisco/DuPont), Axapem™ (Gist-Brocases N.V.), BLAP (sequence shown in FIG. 29 of U.S. Pat. No. 5,352,604) and variants hereof (Henkel AG) and KAP (Bacillus alkalophilus subtilisin) from Kao.

Lipases and Cutinases:

Suitable lipases and cutinases include those of bacterial or fungal origin. Chemically modified or protein engineered mutant enzymes are included. Examples include lipase from Thermomyces, e.g. from T. lanuginosus (previously named Humicola lanuginosa) as described in EP258068 and EP305216, cutinase from Humicola, e.g. H. insolens (WO96/13580), lipase from strains of Pseudomonas (some of these now renamed to Burkholderia), e.g. P. alcaligenes or P. pseudoalcaligenes (EP218272), P. cepacia (EP331376), P. sp. strain SD705 (WO95/06720 & WO96/27002), P. wisconsinensis (WO96/12012), GDSL-type Streptomyces lipases (WO10/065455), cutinase from Magnaporthe grisea (WO10/107560), cutinase from Pseudomonas mendocina (U.S. Pat. No. 5,389,536), lipase from Thermobifida fusca (WO11/084412), Geobacillus stearothermophilus lipase (WO11/084417), lipase from Bacillus subtilis (WO11/084599), and lipase from Streptomyces griseus (WO11/150157) and S. pristinaespiralis (WO12/137147).

Other examples are lipase variants such as those described in EP407225, WO92/05249, WO94/01541, WO94/25578, WO95/14783, WO95/30744, WO95/35381, WO95/22615, WO96/00292, WO97/04079, WO97/07202, WO00/34450, WO00/60063, WO01/92502, WO07/87508 and WO09/109500.

Preferred commercial lipase products include include Lipolase™, Lipex™; Lipolex™ and Lipoclean™ (Novozymes NS), Lumafast (originally from Genencor) and Lipomax (originally from Gist-Brocades).

Still other examples are lipases sometimes referred to as acyltransferases or perhydrolases, e.g. acyltransferases with homology to Candida antarctica lipase A (WO10/111143), acyltransferase from Mycobacterium smegmatis (WO05/56782), perhydrolases from the CE 7 family (WO09/67279), and variants of the M. smegmatis perhydrolase in particular the S54V variant used in the commercial product Gentle Power Bleach from Huntsman Textile Effects Pte Ltd (WO10/100028).

Amylases:

Suitable amylases which can be used together with the polypeptides of the invention may be an alpha-amylase or a glucoamylase and may be of bacterial or fungal origin. Chemically modified or protein engineered mutants are included. Amylases include, for example, alpha-amylases obtained from Bacillus, e.g., a special strain of Bacillus licheniformis, described in more detail in GB 1,296,839.

Suitable amylases include amylases having SEQ ID NO: 2 in WO 95/10603 or variants having 90% sequence identity to SEQ ID NO: 3 thereof. Preferred variants are described in WO 94/02597, WO 94/18314, WO 97/43424 and SEQ ID NO: 4 of WO 99/019467, such as variants with substitutions in one or more of the following positions: 15, 23, 105, 106, 124, 128, 133, 154, 156, 178, 179, 181, 188, 190, 197, 201, 202, 207, 208, 209, 211, 243, 264, 304, 305, 391, 408, and 444.

Different suitable amylases include amylases having SEQ ID NO: 6 in WO 02/010355 or variants thereof having 90% sequence identity to SEQ ID NO: 6. Preferred variants of SEQ ID NO: 6 are those having a deletion in positions 181 and 182 and a substitution in position 193.

Other amylases which are suitable are hybrid alpha-amylase comprising residues 1-33 of the alpha-amylase derived from B. amyloliquefaciens shown in SEQ ID NO: 6 of WO 2006/066594 and residues 36-483 of the B. licheniformis alpha-amylase shown in SEQ ID NO: 4 of WO 2006/066594 or variants having 90% sequence identity thereof. Preferred variants of this hybrid alpha-amylase are those having a substitution, a deletion or an insertion in one of more of the following positions: G48, T49, G107, H156, A181, N190, M197, 1201, A209 and Q264. Most preferred variants of the hybrid alpha-amylase comprising residues 1-33 of the alpha-amylase derived from B. amyloliquefaciens shown in SEQ ID NO: 6 of WO 2006/066594 and residues 36-483 of SEQ ID NO: 4 are those having the substitutions:

M197T;

H156Y+A181T+N190F+A209V+Q264S; or

G48A+T491+G107A+H156Y+A181T+N190F+1201F+A209V+Q264S.

Further amylases which are suitable are amylases having SEQ ID NO: 6 in WO 99/019467 or variants thereof having 90% sequence identity to SEQ ID NO: 6. Preferred variants of SEQ ID NO: 6 are those having a substitution, a deletion or an insertion in one or more of the following positions: R181, G182, H183, G184, N195, 1206, E212, E216 and K269. Particularly preferred amylases are those having deletion in positions R181 and G182, or positions H183 and G184.

Additional amylases which can be used are those having SEQ ID NO: 1, SEQ ID NO: 3, SEQ ID NO: 2 or SEQ ID NO: 7 of WO 96/023873 or variants thereof having 90% sequence identity to SEQ ID NO: 1, SEQ ID NO: 2, SEQ ID NO: 3 or SEQ ID NO: 7. Preferred variants of SEQ ID NO: 1, SEQ ID NO: 2, SEQ ID NO: 3 or SEQ ID NO: 7 are those having a substitution, a deletion or an insertion in one or more of the following positions: 140, 181, 182, 183, 184, 195, 206, 212, 243, 260, 269, 304 and 476, using SEQ ID 2 of WO 96/023873 for numbering. More preferred variants are those having a deletion in two positions selected from 181, 182, 183 and 184, such as 181 and 182, 182 and 183, or positions 183 and 184. Most preferred amylase variants of SEQ ID NO: 1, SEQ ID NO: 2 or SEQ ID NO: 7 are those having a deletion in positions 183 and 184 and a substitution in one or more of positions 140, 195, 206, 243, 260, 304 and 476.

Other amylases which can be used are amylases having SEQ ID NO: 2 of WO 08/153815, SEQ ID NO: 10 in WO 01/66712 or variants thereof having 90% sequence identity to SEQ ID NO: 2 of WO 08/153815 or 90% sequence identity to SEQ ID NO: 10 in WO 01/66712. Preferred variants of SEQ ID NO: 10 in WO 01/66712 are those having a substitution, a deletion or an insertion in one of more of the following positions: 176, 177, 178, 179, 190, 201, 207, 211 and 264.

Further suitable amylases are amylases having SEQ ID NO: 2 of WO 09/061380 or variants having 90% sequence identity to SEQ ID NO: 2 thereof. Preferred variants of SEQ ID NO: 2 are those having a truncation of the C-terminus and/or a substitution, a deletion or an insertion in one of more of the following positions: Q87, Q98, S125, N128, T131, T165, K178, R180, S181, T182, G183, M201, F202, N225, S243, N272, N282, Y305, R309, D319, Q320, Q359, K444 and G475. More preferred variants of SEQ ID NO: 2 are those having the substitution in one of more of the following positions: Q87E,R, Q98R, S125A, N128C, T131I, T165I, K178L, T182G, M201L, F202Y, N225E,R, N272E,R, S243Q,A,E,D, Y305R, R309A, Q320R, Q359E, K444E and G475K and/or deletion in position R180 and/or S181 or of T182 and/or G183. Most preferred amylase variants of SEQ ID NO: 2 are those having the substitutions:

N128C+K178L+T182G+Y305R+G475K;

N128C+K178L+T182G+F202Y+Y305R+D319T+G475K;

S125A+N128C+K178L+T182G+Y305R+G475K; or

S125A+N128C+T131I+T165I+K178L+T182G+Y305R+G475K wherein the variants are C-terminally truncated and optionally further comprises a substitution at position 243 and/or a deletion at position 180 and/or position 181.

Further suitable amylases are amylases having SEQ ID NO: 1 of WO13184577 or variants having 90% sequence identity to SEQ ID NO: 1 thereof. Preferred variants of SEQ ID NO: 1 are those having a substitution, a deletion or an insertion in one of more of the following positions: K176, R178, G179, T180, G181, E187, N192, M199, 1203, S241, R458, T459, D460, G476 and G477. More preferred variants of SEQ ID NO: 1 are those having the substitution in one of more of the following positions: K176L, E187P, N192FYH, M199L, I203YF, S241QADN, R458N, T459S, D460T, G476K and G477K and/or deletion in position R178 and/or S179 or of T180 and/or G181. Most preferred amylase variants of SEQ ID NO: 1 are those having the substitutions:

E187P+I203Y+G476K

E187P+I203Y+R458N+T459S+D460T+G476K

wherein the variants optionally further comprise a substitution at position 241 and/or a deletion at position 178 and/or position 179.

Further suitable amylases are amylases having SEQ ID NO: 1 of WO10104675 or variants having 90% sequence identity to SEQ ID NO: 1 thereof. Preferred variants of SEQ ID NO: 1 are those having a substitution, a deletion or an insertion in one of more of the following positions: N21, D97, V128 K177, R179, S180, I181, G182, M200, L204, E242, G477 and G478. More preferred variants of SEQ ID NO: 1 are those having the substitution in one of more of the following positions: N21 D, D97N, V128I K177L, M200L, L204YF, E242QA, G477K and G478K and/or deletion in position R179 and/or S180 or of I181 and/or G182. Most preferred amylase variants of SEQ ID NO: 1 are those having the substitutions:

N21 D+D97N+V128I

wherein the variants optionally further comprise a substitution at position 200 and/or a deletion at position 180 and/or position 181.

Other suitable amylases are the alpha-amylase having SEQ ID NO: 12 in WO01/66712 or a variant having at least 90% sequence identity to SEQ ID NO: 12. Preferred amylase variants are those having a substitution, a deletion or an insertion in one of more of the following positions of SEQ ID NO: 12 in WO01/66712: R28, R118, N174; R181, G182, D183, G184, G186, W189, N195, M202, Y298, N299, K302, S303, N306, R310, N314; R320, H324, E345, Y396, R400, W439, R444, N445, K446, Q449, R458, N471, N484. Particular preferred amylases include variants having a deletion of D183 and G184 and having the substitutions R118K, N195F, R320K and R458K, and a variant additionally having substitutions in one or more position selected from the group: M9, G149, G182, G186, M202, T257, Y295, N299, M323, E345 and A339, most preferred a variant that additionally has substitutions in all these positions.

Other examples are amylase variants such as those described in WO2011/098531, WO2013/001078 and WO2013/001087.

Commercially available amylases are Duramyl™, Termamyl™, Fungamyl™, Stainzyme™ Stainzyme Plus™, Natalase™, Liquozyme X and BAN™ (from Novozymes NS), and Rapidase™, Purastar™/Effectenz™, Powerase, Preferenz S1000, Preferenz S100 and Preferenz S110 (from Genencor International Inc./DuPont).

Peroxidases/Oxidases

A peroxidase according to the invention is a peroxidase enzyme comprised by the enzyme classification EC 1.11.1.7, as set out by the Nomenclature Committee of the International Union of Biochemistry and Molecular Biology (IUBMB), or any fragment derived therefrom, exhibiting peroxidase activity.

Suitable peroxidases include those of plant, bacterial or fungal origin. Chemically modified or protein engineered mutants are included. Examples of useful peroxidases include peroxidases from Coprinopsis, e.g., from C. cinerea (EP 179,486), and variants thereof as those described in WO 93/24618, WO 95/10602, and WO 98/15257.

A peroxidase according to the invention also include a haloperoxidase enzyme, such as chloroperoxidase, bromoperoxidase and compounds exhibiting chloroperoxidase or bromoperoxidase activity. Haloperoxidases are classified according to their specificity for halide ions. Chloroperoxidases (E.C. 1.11.1.10) catalyze formation of hypochlorite from chloride ions.

In an embodiment, the haloperoxidase of the invention is a chloroperoxidase. Preferably, the haloperoxidase is a vanadium haloperoxidase, i.e., a vanadate-containing haloperoxidase. In a preferred method of the present invention the vanadate-containing haloperoxidase is combined with a source of chloride ion.

Haloperoxidases have been isolated from many different fungi, in particular from the fungus group dematiaceous hyphomycetes, such as Caldariomyces, e.g., C. fumago, Alternaria, Curvularia, e.g., C. verruculosa and C. inaequalis, Drechslera, Ulocladium and Botrytis.

Haloperoxidases have also been isolated from bacteria such as Pseudomonas, e.g., P. pyrrocinia and Streptomyces, e.g., S. aureofaciens.

In an preferred embodiment, the haloperoxidase is derivable from Curvularia sp., in particular Curvularia verruculosa or Curvularia inaequalis, such as C. inaequalis CBS 102.42 as described in WO 95/27046; or C. verruculosa CBS 147.63 or C. verruculosa CBS 444.70 as described in WO 97/04102; or from Drechslera hartlebii as described in WO 01/79459, Dendryphiella salina as described in WO 01/79458, Phaeotrichoconis crotalarie as described in WO 01/79461, or Geniculosporium sp. as described in WO 01/79460.

An oxidase according to the invention include, in particular, any laccase enzyme comprised by the enzyme classification EC 1.10.3.2, or any fragment derived therefrom exhibiting laccase activity, or a compound exhibiting a similar activity, such as a catechol oxidase (EC 1.10.3.1), an o-aminophenol oxidase (EC 1.10.3.4), or a bilirubin oxidase (EC 1.3.3.5).

Preferred laccase enzymes are enzymes of microbial origin. The enzymes may be derived from plants, bacteria or fungi (including filamentous fungi and yeasts).

Suitable examples from fungi include a laccase derivable from a strain of Aspergillus, Neurospora, e.g., N. crassa, Podospora, Botrytis, Collybia, Fomes, Lentinus, Pleurotus, Trametes, e.g., T. villosa and T. versicolor, Rhizoctonia, e.g., R. solani, Coprinopsis, e.g., C. cinerea, C. comatus, C. friesii, and C. plicatilis, Psathyrella, e.g., P. condelleana, Panaeolus, e.g., P. papilionaceus, Myceliophthora, e.g., M. thermophila, Schytalidium, e.g., S. thermophilum, Polyporus, e.g., P. pinsitus, Phlebia, e.g., P. radiata (WO 92/01046), or Coriolus, e.g., C. hirsutus (JP 2238885).

Suitable examples from bacteria include a laccase derivable from a strain of Bacillus.

A laccase derived from Coprinopsis or Myceliophthora is preferred; in particular a laccase derived from Coprinopsis cinerea, as disclosed in WO 97/08325; or from Myceliophthora thermophila, as disclosed in WO 95/33836.

Nucleases

Suitable nucleases include deoxyribonucleases (DNases) as well as ribonucleases. DNases are any enzyme that catalyzes the hydrolytic cleavage of phosphodiester linkages in the DNA backbone, thus degrading DNA. According to the invention, a DNase which is obtainable from a bacterium is preferred; in particular a DNase, which is obtainable from a Bacillus is preferred; in particular a DNase which is obtainable from Bacillus subtilis or Bacillus licheniformis is preferred. Examples of such DNases are described in patent application WO 2011/098579 or in PCT/EP2013/075922.

The detergent enzyme(s) may be included in a detergent composition by adding separate additives containing one or more enzymes, or by adding a combined additive comprising all of these enzymes. A detergent additive of the invention, i.e., a separate additive or a combined additive, can be formulated, for example, as a granulate, liquid, slurry, etc. Preferred detergent additive formulations are granulates, in particular non-dusting granulates, liquids, in particular stabilized liquids, or slurries.

Non-dusting granulates may be produced, e.g. as disclosed in U.S. Pat. Nos. 4,106,991 and 4,661,452 and may optionally be coated by methods known in the art. Examples of waxy coating materials are polyethyleneglycol (PEG) with mean molar weights of 1000 to 20000; ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in GB 1483591. Liquid enzyme preparations may, for instance, be stabilized by adding a polyol such as propylene glycol, a sugar or sugar alcohol, lactic acid or boric acid according to established methods. Protected enzymes may be prepared according to the method disclosed in EP 238,216.

Microorganisms

The detergent additive as well as the detergent composition may also comprise one or more microorganisms, such as one or more fungi, yeast, or bacteria.

In an embodiment, the one or more microorganisms are dehydrated (for example by lyophilization) bacteria or yeast, such as a strain of Lactobacillus.

In another embodiment, the microorganisms are one or more microbial spores (as opposed to vegetative cells), such as bacterial spores; or fungal spores, conidia, hypha. Preferably, the one or more spores are Bacillus endospores; even more preferably the one or more spores are endospores of Bacillus subtilis, Bacillus licheniformis, Bacillus amyloliquefaciens, or Bacillus megaterium.

The microorganisms may be included in the detergent composition or additive in the same way as enzymes (see above).

### Adjunct Materials

Any detergent components known in the art for use in laundry detergents may also be utilized. Other optional detergent components include anti-corrosion agents, anti-shrink agents, antisoil redeposition agents, anti-wrinkling agents, bactericides, binders, corrosion inhibitors, disintegrants/disintegration agents, dyes, enzyme stabilizers (including boric acid, borates, CMC, and/or polyols such as propylene glycol), fabric conditioners including clays, fillers/processing aids, fluorescent whitening agents/optical brighteners, foam boosters, foam (suds) regulators, perfumes, soil-suspending agents, softeners, suds suppressors, tarnish inhibitors, and wicking agents, either alone or in combination. Any ingredient known in the art for use in laundry detergents may be utilized. The choice of such ingredients is well within the skill of the artisan.

### Dispersants

The detergent compositions of the present invention can also contain dispersants. In particular powdered detergents may comprise dispersants. Suitable water-soluble organic materials include the homo- or co-polymeric acids or their salts, in which the polycarboxylic acid comprises at least two carboxyl radicals separated from each other by not more than two carbon atoms. Suitable dispersants are for example described in Powdered Detergents, Surfactant science series volume 71, Marcel Dekker, Inc.

### Dye Transfer Inhibiting Agents

The detergent compositions of the present invention may also include one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. When present in a subject composition, the dye transfer inhibiting agents may be present at levels from about 0.0001% to about 10%, from about 0.01% to about 5% or even from about 0.1% to about 3% by weight of the composition.

### Fluorescent Whitening Agent

The detergent compositions of the present invention will preferably also contain additional components that may tint articles being cleaned, such as fluorescent whitening agent or optical brighteners. Where present the brightener is preferably at a level of about 0.01% to about 0.5%. Any fluorescent whitening agent suitable for use in a laundry detergent composition may be used in the composition of the present invention. The most commonly used fluorescent whitening agents are those belonging to the classes of diaminostilbene-sulfonic acid derivatives, diarylpyrazoline derivatives and bisphenyl-distyryl derivatives. Examples of the diaminostilbene-sulfonic acid derivative type of fluorescent whitening agents include the sodium salts of: 4,4′-bis-(2-diethanolamino-4-anilino-s-triazin-6-ylamino) stilbene-2,2′-disulfonate, 4,4′-bis-(2,4-dianilino-s-triazin-6-ylamino) stilbene-2.2′-disulfonate, 4,4′-bis-(2-anilino-4-(N-methyl-N-2-hydroxy-ethylamino)-s-triazin-6-ylamino) stilbene-2,2′-disulfonate, 4,4′-bis-(4-phenyl-1,2,3-triazol-2-yl)stilbene-2,2′-disulfonate and sodium 5-(2H-naphtho[1,2-d][1,2,3]triazol-2-yl)-2-[(E)-2-phenylvinyl]benzenesulfonate. Preferred fluorescent whitening agents are Tinopal DMS and Tinopal CBS available from Ciba-Geigy AG, Basel, Switzerland. Tinopal DMS is the disodium salt of 4,4′-bis-(2-morpholino-4-anilino-s-triazin-6-ylamino) stilbene-2,2′-disulfonate. Tinopal CBS is the disodium salt of 2,2′-bis-(phenyl-styryl)-disulfonate. Also preferred are fluorescent whitening agents is the commercially available Parawhite KX, supplied by Paramount Minerals and Chemicals, Mumbai, India. Other fluorescers suitable for use in the invention include the 1-3-diaryl pyrazolines and the 7-alkylaminocoumarins.

Suitable fluorescent brightener levels include lower levels of from about 0.01, from 0.05, from about 0.1 or even from about 0.2 wt % to upper levels of 0.5 or even 0.75 wt %.

### Soil Release Polymers

The detergent compositions of the present invention may also include one or more soil release polymers which aid the removal of soils from fabrics such as cotton and polyester based fabrics, in particular the removal of hydrophobic soils from polyester based fabrics. The soil release polymers may for example be nonionic or anionic terephthalte based polymers, polyvinyl caprolactam and related copolymers, vinyl graft copolymers, polyester polyamides see for example Chapter 7 in Powdered Detergents, Surfactant science series volume 71, Marcel Dekker, Inc. Another type of soil release polymers are amphiphilic alkoxylated grease cleaning polymers comprising a core structure and a plurality of alkoxylate groups attached to that core structure. The core structure may comprise a polyalkylenimine structure or a polyalkanolamine structure as described in detail in WO 2009/087523 (hereby incorporated by reference). Furthermore random graft co-polymers are suitable soil release polymers. Suitable graft co-polymers are described in more detail in WO 2007/138054, WO 2006/108856 and WO 2006/113314 (hereby incorporated by reference). Other soil release polymers are substituted polysaccharide structures especially substituted cellulosic structures such as modified cellulose deriviatives such as those described in EP 1867808 or WO 2003/040279 (both are hereby incorporated by reference). Suitable cellulosic polymers include cellulose, cellulose ethers, cellulose esters, cellulose amides and mixtures thereof. Suitable cellulosic polymers include anionically modified cellulose, nonionically modified cellulose, cationically modified cellulose, zwitterionically modified cellulose, and mixtures thereof. Suitable cellulosic polymers include methyl cellulose, carboxy methyl cellulose, ethyl cellulose, hydroxyl ethyl cellulose, hydroxyl propyl methyl cellulose, ester carboxy methyl cellulose, and mixtures thereof.

### Anti-Redeposition Agents

The detergent compositions of the present invention may also include one or more antiredeposition agents such as carboxymethylcellulose (CMC), polyvinyl alcohol (PVA), polyvinylpyrrolidone (PVP), polyoxyethylene and/or polyethyleneglycol (PEG), homopolymers of acrylic acid, copolymers of acrylic acid and maleic acid, and ethoxylated polyethyleneimines. The cellulose based polymers described under soil release polymers above may also function as antiredeposition agents.

### Rheology Modifiers

The detergent compositions of the present invention may also include one or more rheology modifiers, structurants or thickeners, as distinct from viscosity reducing agents. The rheology modifiers are selected from the group consisting of non-polymeric crystalline, hydroxy-functional materials, polymeric rheology modifiers which impart shear thinning characteristics to the aqueous liquid matrix of a liquid detergent composition. The rheology and viscosity of the detergent can be modified and adjusted by methods known in the art, for example as shown in EP 2169040.

Other suitable adjunct materials include, but are not limited to, anti-shrink agents, anti-wrinkling agents, bactericides, binders, carriers, dyes, enzyme stabilizers, fabric softeners, fillers, foam regulators, hydrotropes, perfumes, pigments, sod suppressors, solvents, and structurants for liquid detergents and/or structure elasticizing agents.

### Formulation of Detergent Products

The detergent composition of the invention may be in any convenient form, e.g., a bar, a homogenous tablet, a tablet having two or more layers, a pouch having one or more compartments, a regular or compact powder, a granule, a paste, a gel, or a regular, compact or concentrated liquid.

Pouches can be configured as single or multicompartments. It can be of any form, shape and material which is suitable for hold the composition, e.g. without allowing the release of the composition to release of the composition from the pouch prior to water contact. The pouch is made from water soluble film which encloses an inner volume. Said inner volume can be divided into compartments of the pouch. Preferred films are polymeric materials preferably polymers which are formed into a film or sheet. Preferred polymers, copolymers or derivates thereof are selected polyacrylates, and water soluble acrylate copolymers, methyl cellulose, carboxy methyl cellulose, sodium dextrin, ethyl cellulose, hydroxyethyl cellulose, hydroxypropyl methyl cellulose, malto dextrin, poly methacrylates, most preferably polyvinyl alcohol copolymers and, hydroxypropyl methyl cellulose (HPMC). Preferably the level of polymer in the film for example PVA is at least about 60%. Preferred average molecular weight will typically be about 20,000 to about 150,000. Films can also be of blended compositions comprising hydrolytically degradable and water soluble polymer blends such as polylactide and polyvinyl alcohol (known under the Trade reference M8630 as sold by MonoSol LLC, Indiana, USA) plus plasticisers like glycerol, ethylene glycerol, propylene glycol, sorbitol and mixtures thereof. The pouches can comprise a solid laundry cleaning composition or part components and/or a liquid cleaning composition or part components separated by the water soluble film. The compartment for liquid components can be different in composition than compartments containing solids: US2009/0011970 A1.

Detergent ingredients can be separated physically from each other by compartments in water dissolvable pouches or in different layers of tablets. Thereby negative storage interaction between components can be avoided. Different dissolution profiles of each of the compartments can also give rise to delayed dissolution of selected components in the wash solution.

A liquid or gel detergent, which is not unit dosed, may be aqueous, typically containing at least 20% by weight and up to 95% water, such as up to about 70% water, up to about 65% water, up to about 55% water, up to about 45% water, up to about 35% water. Other types of liquids, including without limitation, alkanols, amines, diols, ethers and polyols may be included in an aqueous liquid or gel. An aqueous liquid or gel detergent may contain from 0-30% organic solvent.

A liquid or gel detergent may be non-aqueous.

### Laundry Soap Bars

The polypeptides of the invention may be added to laundry soap bars and used for hand washing laundry, fabrics and/or textiles. The term laundry soap bar includes laundry bars, soap bars, combo bars, syndet bars and detergent bars. The types of bar usually differ in the type of surfactant they contain, and the term laundry soap bar includes those containing soaps from fatty acids and/or synthetic soaps. The laundry soap bar has a physical form which is solid and not a liquid, gel or a powder at room temperature. The term solid is defined as a physical form which does not significantly change over time, i.e. if a solid object (e.g. laundry soap bar) is placed inside a container, the solid object does not change to fill the container it is placed in. The bar is a solid typically in bar form but can be in other solid shapes such as round or oval.

The laundry soap bar may contain one or more additional enzymes, protease inhibitors such as peptide aldehydes (or hydrosulfite adduct or hemiacetal adduct), boric acid, borate, borax and/or phenylboronic acid derivatives such as 4-formylphenylboronic acid, one or more soaps or synthetic surfactants, polyols such as glycerine, pH controlling compounds such as fatty acids, citric acid, acetic acid and/or formic acid, and/or a salt of a monovalent cation and an organic anion wherein the monovalent cation may be for example Na⁺, K⁺ or NH₄⁺ and the organic anion may be for example formate, acetate, citrate or lactate such that the salt of a monovalent cation and an organic anion may be, for example, sodium formate.

The laundry soap bar may also contain complexing agents like EDTA and HEDP, perfumes and/or different type of fillers, surfactants e.g. anionic synthetic surfactants, builders, polymeric soil release agents, detergent chelators, stabilizing agents, fillers, dyes, colorants, dye transfer inhibitors, alkoxylated polycarbonates, suds suppressers, structurants, binders, leaching agents, bleaching activators, clay soil removal agents, anti-redeposition agents, polymeric dispersing agents, brighteners, fabric softeners, perfumes and/or other compounds known in the art.

The laundry soap bar may be processed in conventional laundry soap bar making equipment such as but not limited to: mixers, plodders, e.g. a two stage vacuum plodder, extruders, cutters, logo-stampers, cooling tunnels and wrappers. The invention is not limited to preparing the laundry soap bars by any single method. The premix of the invention may be added to the soap at different stages of the process. For example, the premix containing a soap, polypeptide of the invention optionally one or more additional enzymes, a protease inhibitor, and a salt of a monovalent cation and an organic anion may be prepared and the mixture is then plodded. The polypeptides of the invention and optional additional enzymes may be added at the same time as the protease inhibitor for example in liquid form. Besides the mixing step and the plodding step, the process may further comprise the steps of milling, extruding, cutting, stamping, cooling and/or wrapping.

### Granular Detergent Formulations

A granular detergent may be formulated as described in WO09/092699, EP1705241, EP1382668, WO07/001262, U.S. Pat. No. 6,472,364, WO04/074419 or WO09/102854. Other useful detergent formulations are described in WO09/124162, WO09/124163, WO09/117340, WO09/117341, WO09/117342, WO09/072069, WO09/063355, WO09/132870, WO09/121757, WO09/112296, WO09/112298, WO09/103822, WO09/087033, WO09/050026, WO09/047125, WO09/047126, WO09/047127, WO09/047128, WO09/021784, WO09/010375, WO09/000605, WO09/122125, WO09/095645, WO09/040544, WO09/040545, WO09/024780, WO09/004295, WO09/004294, WO09/121725, WO09/115391, WO09/115392, WO09/074398, WO09/074403, WO09/068501, WO09/065770, WO09/021813, WO09/030632, and WO09/015951.

WO2011025615, WO2011016958, WO2011005803, WO2011005623, WO2011005730, WO2011005844, WO2011005904, WO2011005630, WO2011005830, WO2011005912, WO2011005905, WO2011005910, WO2011005813, WO2010135238, WO2010120863, WO2010108002, WO2010111365, WO2010108000, WO2010107635, WO2010090915, WO2010033976, WO2010033746, WO2010033747, WO2010033897, WO2010033979, WO2010030540, WO2010030541, WO2010030539, WO2010024467, WO2010024469, WO2010024470, WO2010025161, WO2010014395, WO2010044905,

WO2010145887, WO2010142503, WO2010122051, WO2010102861, WO2010099997, WO2010084039, WO2010076292, WO2010069742, WO2010069718, WO2010069957, WO2010057784, WO2010054986, WO2010018043, WO2010003783, WO2010003792,

WO2011023716, WO2010142539, WO2010118959, WO2010115813, WO2010105942, WO2010105961, WO2010105962, WO2010094356, WO2010084203, WO2010078979, WO2010072456, WO2010069905, WO2010076165, WO2010072603, WO2010066486, WO2010066631, WO2010066632, WO2010063689, WO2010060821, WO2010049187, WO2010031607, WO2010000636.

### Formulation of Enzyme in Co-Granule

The enzyme of the invention may be formulated as a granule for example as a co-granule that combines one or more enzymes. Each enzyme will then be present in more granules securing a more uniform distribution of enzymes in the detergent. This also reduces the physical segregation of different enzymes due to different particle sizes. Methods for producing multi-enzyme cogranulates for the detergent industry are disclosed in the IP.com disclosure IPCOM000200739D.

Another example of formulation of enzymes by the use of co-granulates are disclosed in WO 2013/188331, which relates to a detergent composition comprising (a) a multi-enzyme cogranule; (b) less than 10 wt zeolite (anhydrous basis); and (c) less than 10 wt phosphate salt (anhydrous basis), wherein said enzyme co-granule comprises from 10 to 98 wt % moisture sink component and the composition additionally comprises from 20 to 80 wt % detergent moisture sink component.

WO 2013/188331 also relates to a method of treating and/or cleaning a surface, preferably a fabric surface comprising the steps of (i) contacting said surface with the detergent composition as claimed and described herein in an aqueous wash liquor, (ii) rinsing and/or drying the surface.

The multi-enzyme co-granule may comprise an enzyme of the invention and (a) one or more enzymes selected from the group consisting of first-wash lipases, cleaning cellulases, xyloglucanases, perhydrolases, peroxidases, lipoxygenases, laccases and mixtures thereof; and (b) one or more enzymes selected from the group consisting of hemicellulases, proteases, care cellulases, cellobiose dehydrogenases, xylanases, phospho lipases, esterases, cutinases, pectinases, mannanases, pectate lyases, keratinases, reductases, oxidases, phenoloxidases, ligninases, pullulanases, tannases, pentosanases, lichenases glucanases, arabinosidases, hyaluronidase, chondroitinase, amylases, nucleases, and mixtures thereof. In another embodiment, the multi-enzyme co-granule does not comprise a cellulase.

### Use in Detergents.

The polypeptides of the present invention may be added to and thus become a component of a detergent composition.

The detergent composition of the present invention may be formulated, for example, as a hand or machine laundry detergent composition including a laundry additive composition suitable for pre-treatment of stained fabrics and a rinse added fabric softener composition, or be formulated as a detergent composition for use in general household hard surface cleaning operations, or be formulated for hand or machine dishwashing operations.

In a specific aspect, the present invention provides a detergent additive comprising a polypeptide of the present invention as described herein.

### EXAMPLES

### Materials and Methods

Evaluation of wrinkles: AATCC (American Association of Textile Chemists and Colorists) test method 124-TM 124 Smoothness Appearance of Fabrics after Home Laundering (available at members.aatcc.org/store/tm124/533/) (AATCC test method TM 124-2018).
Evaluation of static: AATCC test method 115-Electrostatic Clinging of Fabrics: Fabric-to-Metal Test (available at members.aatcc.org/store/tm115/525/).

### Evaluation by Panellist Preference:

Panelists are asked to select T-shirt part being the less creased. After evaluation, distribution is calculated.

The softness and anti-crease is indicated with X:Y values, wherein X specifies the % of the panelists preferring real items washed with CBM, and Y specifies the % that prefers real item washed without CBM. The sum of the X and Y values is 100%.

### Detergent Compositions

The below mentioned detergent composition can be used in combination with the carbohydrate binding modules described herein for preventing or reducing creases and wrinkles in laundry.

### Composition of Model Detergent B (Liquid):

| Ingredient | Amount (wt %) |
|---|---|
| NaOH, pellets (>99%) | 1.05 |
| Linear alkylbenzenesulfonic acid (LAS) (97%) | 7.20 |
| Sodium laureth sulfate (SLES) (28%) | 10.58 |
| Soy fatty acid (>90%) | 2.75 |
| Coco fatty acid (>99%) | 2.75 |
| AEO; alcohol ethoxylate with 8 | 6.60 |
| mol EO; Lutensol TO 8 (~100%) | |
| Triethanol amine (100%) | 3.33 |
| Na-citrate, dihydrate (100%) | 2.00 |
| DTMPA; diethylenetriaminepentakis(methyl- | 0.48 |
| ene)pentakis(phosphonic acid), heptasodium | |
| salt (Dequest 2066 C) (~42% as Na7 salt) | |
| MPG (>98%) | 6.00 |
| EtOH, propan-2-ol (90/10%) | 3.00 |
| Glycerol (>99.5) | 1.71 |
| Sodium formate (>95%) | 1.00 |
| PCA (40% as sodium salt) | 0.46 |
| Water up to | 100 |

Final adjustments to the specified pH (pH 8 in the case of Model Detergent B) were done with NaOH or citric acid. Water hardness was adjusted to 15° dH by addition of CaCl₂) and MgCl₂ (Ca²⁺:Mg²⁺=4:1) to the test system.

Composition of Ariel Sensitive White & Color, liquid detergent composition: Aqua, Alcohol Ethoxy Sulfate, Alcohol Ethoxylate, Amino Oxide, Citrid Acid, C12-18 topped palm kernel fatty acid, Protease, Glycosidase, Amylase, Ethanol, 1,2 Propanediol, Sodium Formate, Calcium Chloride, Sodium hydroxide, Silicone Emulsion, Trans-sulphated EHDQ (the ingredients are listed in descending order).

Composition of WFK IEC-A model detergent (powder): Ingredients: Linear sodium alkyl benzene sulfonate 8,8%, Ethoxylated fatty alcohol 012-18 (7 EO) 4,7%, Sodium soap 3,2%, Anti foam DC2-42485 3,9%, Sodium aluminium silicate zeolite 4A 28,3%, Sodium carbonate 11,6%, Sodium salt of a copolymer from acrylic and maleic acid (Sokalan CP5) 2,4%, Sodium silicate 3,0%, Carboxymethylcellulose 1,2%, Dequest 2066 2,8%, Optical whitener 0,2%, Sodium sulfate6,5%, Protease 0,4%.

Composition of model detergent A (liquid): Ingredients: 12% LAS, 11% AEO Biosoft N25-7 (NI), 7% AEOS (SLES), 6% MPG (monopropylene glycol), 3% ethanol, 3% TEA, 2.75% cocoa soap, 2.75% soya soap, 2% glycerol, 2% sodium hydroxide, 2% sodium citrate, 1% sodium formiate, 0.2% DTMPA and 0.2% PCA (all percentages are w/w)

Composition of Ariel Actilift (liquid): Ingredients: 5-15% Anionic surfactants; <5% Non-ionic surfactants, Phosphonates, Soap; Enzymes, Optical brighteners, Benzisothiazolinone, Methylisothiazolinone, Perfumes, Alpha-isomethyl ionone, Citronellol, Geraniol, Linalool.

Composition of Ariel Actilift Colour & Style (Ariel Colour & Style): Aqua, Sodium Dodecylbenzenesulfonate, C14-C15 Pareth-7, Sodium Citrate, Propylene Glycol, Sodium Palm Kernelate, Sodium Laureth Sulfate, MEA Dodecylbenzenesulfonage, Sulfated Ethoxylated Hexamethylenediamine Quaternized, Sodium Cumenesulfonate, Perfume, Co-polymer of PEG/Vinyl Acetate, Sodium formate, Hydrogenated Castor Oil, Sodium Diethylenetriamine Pentamethylene Phosphonate, PEG/PPG-10/2 Propylheptyl Ether, Butyophenyl Methylpropional, Polyvinylpyridine-N-Oxide, Sorbitol, Glycerin, Ethanolamine, Sodium Hydroxide, Alpha-Isomethyl Ionone, Protease, Calcium Chloride, Geraniol, Linalool, Citronelllol, Tripropylene Glycol, Glycosidase, Benzisothiazolinone, Dimethicone, Glycosidase, Sodium Acetate, Cellulase, Colorant, Glyceryl Stearate, Hydroxyethylcellulose, Silica.

Composition of Ariel Actilift Colour & Style, new pack: Ingredients: Aqua, Sodium Laureth Sulfate, Propylene Glycol, C14-C15 Pareth-7, Sodium citrate, Sodium Palm Kernelate, Alcohol, Sodium Formate, Sulfated Ethoxylated Hexamethylenediamine Quaternized, Sodium Hydroxide, Perfume, Polyvinylpyridine-N-Oxide, Sorbitol, Calcium Chloride, protease, Glycerin, Glucosidase, Glycosidase, Sodium Acetate, Colorant, Cellulase.

Composition of Ariel Actilift Whites & Colours Coolclean, new pack: Ingredients: Aqua, Sodium Laureth Sulfate, Propylene Glycol, C14-C15 Pareth-7, Sodium citrate, Sodium Palm Kernelate, Alcohol, Sodium Formate, Sulfated Ethoxylated Hexamethylenediamine Quaternized, Sodium Hydroxide, Perfume, Sorbitol, Calcium Chloride, protease, Glycerin, Glucosidase, Glycosidase, Sodium Acetate, Colorant, Cellulase.

Composition of Ariel Sensitive White & Color: Ingredients: Aqua, Sodium Laureth Sulfate, Propylene Glycol, C14-C15 Pareth-7, Sodium citrate, Sodium Palm Kernelate, Alcohol, Sodium Formate, Sulfated Ethoxylated Hexamethylenediamine Quaternized, Sodium Hydroxide-Sorbitol, Calcium Chloride, protease, Glycerin, Glycosidase, Sodium Acetate, Cellulase, Silica.

Composition of Ariel Actilift, regular: Aqua, Sodium Dodecylbenzenesulfonate, C14-C15 Pareth-7, Sodium Citrate, Propylene Glycol, Sodium Palm Kernelate, Sodium Laureth Sulfate, MEA Dodecylbenzenesulfonage, Sulfated Ethoxylated Hexamethylenediamine Quaternized, Sodium Cumenesulfonate, Perfume, Co-polymer of PEG/Vinyl Acetate, Sodium formate, C12-C14 Pareth-7, Hydrogenated Castor Oil, Sodium Diethylenetriamine Pentamethylene Phosphonate, PEG/PPG-10/2 Propylheptyl Ether, Butyophenyl Methylpropional, Fluorescent Brightener 9, Sorbitol, Glycerin, Ethanolamine, Sodium Hydroxide, Alpha-Isomethyl Ionone, Protease, Calcium Chloride, Geraniol, Linalool, Citronelllol, Tripropylene Glycol, Sodium Chloride, Glycosidase, Benzisothiazolinone, Dimethicone, Glycosidase, Sodium Acetate, Cellulase, Colorant, Glyceryl Stearate, Hydroxyethylcellulose, Silica.

Composition of Persil Small & Mighty (liquid): Ingredients: 15-30% Anionic surfactants, Non-ionic surfacts, 5-15% Soap, <5% Polycarboxylates, Perfume, Phosphates, Optical Brighteners

Composition of Fairy Non Bio (liquid): Ingredients: 15-30% Anionic Surfactants,5-15% Non-Ionic Surfactants, Soap, Benzisothiazolinone, Methylisothiazolinone, Perfumes

Composition of Model detergent T (powder): Ingredients: 11% LAS, 2% AS/AEOS, 2% soap, 3% AEO, 15.15% sodium carbonate, 3% sodium slilcate, 18.75% zeolite, 0.15% chelant, 2% sodium citrate, 1.65% AA/MA copolymer, 2.5% CMC and 0.5% SRP (all percentages are w/w).

Composition of Model detergent X (powder): Ingredients: 16.5% LAS, 15% zeolite, 12% sodium disilicate, 20% sodium carbonate, 1% sokalan, 35.5% sodium sulfate (all percentages are w/w).

Composition of Ariel Actilift (powder): Ingredients: 15-30% Anionic surfactants, <5% Nonionic surfactants, Phosphonates, Polycarboxylates, Zeolites; Enzymes, Perfumes, Hexyl cinnamal.

Composition of Persil Megaperls (powder): Ingredients: 15-30% of the following: anionic surfactants, oxygen-based bleaching agent and zeolites, less than 5% of the following: non-ionic surfactants, phosphonates, polycarboxylates, soap, Further ingredients: Perfumes, Hexyl cinnamal, Benzyl salicylate, Linalool, optical brighteners, Enzymes and Citronellol.

Gain Liquid, Original: Ingredients: Water, Alcohol Ethoxysulfate, Diethylene Glycol, Alcohol

Ethoxylate, Ethanolamine, Linear Alkyl Benzene Sulfonate, Sodium Fatty Acids, Polyethyleneimine Ethoxylate, Citric Acid, Borax, Sodium Cumene Sulfonate, Propylene Glycol, DTPA, Disodium Diaminostilbene Disulfonate, Dipropylethyl Tetramine, Sodium Hydroxide,

Sodium Formate, Calcium Formate, Dimethicone, Amylase, Protease, Liquitint™, Hydrogenated Castor Oil, Fragrance

Tide Liquid, Original: Ingredients: Linear alkylbenzene sulfonate, propylene glycol, citric acid, sodium hydroxide, borax, ethanolamine, ethanol, alcohol sulfate, polyethyleneimine ethoxylate, sodium fatty acids, diquaternium ethoxysulfate, protease, diethylene glycol, laureth-9, alkyldimethylamine oxide, fragrance, amylase, disodium diaminostilbene disulfonate, DTPA, sodium formate, calcium formate, polyethylene glycol 4000, mannanase, Liquitint™ Blue, dimethicone.

Liquid Tide, Free and Gentle: Water, sodium alcoholethoxy sulfate, propylene glycol, borax, ethanol, linear alkylbenzene sulfonate sodium, salt, polyethyleneimine ethoxylate, diethylene glycol, trans sulfated & ethoxylated hexamethylene diamine, alcohol ethoxylate, linear alkylbenzene sulfonate, MEA salt, sodium formate, sodium alkyl sulfate, DTPA, amine oxide, calcium formate, disodium diaminostilbene, disulfonate, amylase, protease, dimethicone, benzisothiazolinone

Tide Coldwater Liquid, Fresh Scent: Water, alcoholethoxy sulfate, linear alkylbenzene sulfonate, diethylene glycol, propylene glycol, ethanolamine, citric acid, Borax, alcohol sulfate, sodium hydroxide, polyethyleneimine, ethoxylate, sodium fatty acids, ethanol, protease, Laureth-9, diquaternium ethoxysulfate, lauramine oxide, sodium cumene, sulfonate, fragrance, DTPA, amylase, disodium, diaminostilbene, disulfonate, sodium formate, disodium distyrylbiphenyl disulfonate, calcium formate, polyethylene glycol 4000, mannanase, pectinase, Liquitint™ Blue, dimethicone

Tide TOTALCARE™ Liquid, Cool Cotton: Water, alcoholethoxy sulfate, propylene glycol, sodium fatty acids, laurtrimonium chloride, ethanol, sodium hydroxide, sodium cumene sulfonate, citric acid, ethanolamine, diethylene glycol, silicone polyether, borax, fragrance, polyethyleneimine ethoxylate, protease, Laureth-9, DTPA, polyacrylamide quaternium chloride, disodium diaminostilbene disulfonate, sodium formate, Liquitint™ Orange, dipropylethyl tetraamine, dimethicone, cellulase,

Liquid Tide Plus Bleach Alternative™, Vivid White and Bright, Original and Clean Breeze: Water, sodium alcoholethoxy sulfate, sodium alkyl sulfate, MEA citrate, linear alkylbenzene sulfonate, MEA salt, propylene glycol, diethylene glycol, polyethyleneimine ethoxylate, ethanol, sodium fatty acids, ethanolamine, lauramine oxide, borax, Laureth-9, DTPA, sodium cumene sulfonate, sodium formate, calcium formate, linear alkylbenzene sulfonate, sodium salt, alcohol sulfate, sodium hydroxide, diquaternium ethoxysulfate, fragrance, amylase, protease, mannanase, pectinase, disodium diaminostilbene disulfonate, benzisothiazolinone, Liquitint™ Blue, dimethicone, dipropylethyl tetraamine.

Liquid Tide HE, Original Scent: Water, Sodium alcoholethoxy sulfate, MEA citrate, Sodium Alkyl Sulfate, alcohol ethoxylate, linear alkylbenzene sulfonate, MEA salt, sodium fatty acids, polyethyleneimine ethoxylate, diethylene glycol, propylene glycol, diquaternium ethoxysulfate, borax, polyethyleneimine, ethoxylate propoxylate, ethanol, sodium cumene sulfonate, fragrance, DTPA, disodium diaminostilbene disulfonate, Mannanase, cellulase, amylase, sodium formate, calcium formate, Lauramine oxide, Liquitint™ Blue, Dimethicone/polydimethyl silicone.

Tide TOTALCARE HE Liquid, renewing Rain: Water, alcoholethoxy sulfate, linear alkylbenzene sulfonate, alcohol ethoxylate, citric acid, Ethanolamine, sodium fatty acids, diethylene glycol, propylene glycol, sodium hydroxide, borax, polyethyleneimine ethoxylate, silicone polyether, ethanol, protease, sodium cumene sulfonate, diquaternium ethoxysulfate, Laureth-9, fragrance, amylase, DTPA, disodium diaminostilbene disulfonate, disodium distyrylbiphenyl disulfonate, sodium formate, calcium formate, mannanase, Liquitint™ Orange, dimethicone, polyacrylamide quaternium chloride, cellulase, dipropylethyl tetraamine.

Tide liquid HE Free: Water, alcoholethoxy sulfate, diethylene glycol, monoethanolamine citrate, sodium formate, propylene glycol, linear alkylbenzene sulfonates, ethanolamine, ethanol, polyethyleneimine ethoxylate, amylase, benzisothiazolin, borax, calcium formate, citric acid, diethylenetriamine pentaacetate sodium, dimethicone, diquaternium ethoxysulfate, disodium diaminostilbene disulfonate, Laureth-9, mannanase, protease, sodium cumene sulfonate, sodium fatty acids.

Tide Coldwater HE Liquid, Fresh Scent: Water, alcoholethoxy sulfate, MEA Citrate, alcohol sulfate, Alcohol ethoxylate, Linear alkylbenzene sulfonate MEA, sodium fatty acids, polyethyleneimine ethoxylate, diethylene glycol, propylene glycol, diquaternium ethoxysulfate, borax, polyethyleneimine ethoxylate propoxylate, ethanol, sodium cumene sulfonate, fragrance, DTPA, disodium diaminostilbene disulfonate, protease, mannanase, cellulase, amylase, sodium formate, calcium formate, lauramine oxide, Liquitint™ Blue, dimethicone.

Tide for Coldwater HE Free Liquid: Water, sodium alcoholethoxy sulfate, MEA Citrate, Linear alkylbenzene sulfonate: sodium salt, Alcohol ethoxylate, Linear alkylbenzene sulfonate: MEA salt, sodium fatty acids, polyethyleneimine ethoxylate, diethylene glycol, propylene glycol, diquaternium ethoxysulfate, Borax, protease, polyethyleneimine ethoxylate propoxylate, ethanol, sodium cumene sulfonate, Amylase, citric acid, DTPA, disodium diaminostilbene disulfonate, sodium formate, calcium formate, dimethicone.

Tide Simply Clean & Fresh: Water, alcohol ethoxylate sulfate, linear alkylbenzene sulfonate Sodium/Mea salts, propylene glycol, diethylene glycol, sodium formate, ethanol, borax, sodium fatty acids, fragrance, lauramine oxide, DTPA, Polyethylene amine ethoxylate, calcium formate, disodium diaminostilbene disulfonate, dimethicone, tetramine, Liquitint™ Blue.

Tide Pods, Ocean Mist, Mystic Forest, Spring Meadow: Linear alkylbenzene sulfonates, C12-16 Pareth-9, propylene glycol, alcoholethoxy sulfate, water, polyethyleneimine ethoxylate, glycerine, fatty acid salts, PEG-136 polyvinyl acetate, ethylene Diamine disuccinic salt, monoethanolamine citrate, sodium bisulfite, diethylenetriamine pentaacetate sodium, disodium distyrylbiphenyl disulfonate, calcium formate, mannanase, exyloglucanase, sodium formate, hydrogenated castor oil, natalase, dyes, termamyl, subtilisin, benzisothiazolin, perfume.

Tide to Go: Deionized water, Dipropylene Glycol Butyl Ether, Sodium Alkyl Sulfate, Hydrogen Peroxide, Ethanol, Magnesium Sulfate, Alkyl Dimethyl Amine Oxide, Citric Acid, Sodium Hydroxide, Trimethoxy Benzoic Acid, Fragrance.

Tide Stain Release Liquid: Water, Alkyl Ethoxylate, Linear Alkylbenzenesulfonate, Hydrogen Peroxide, Diquaternium Ethoxysulfate, Ethanolamine, Disodium Distyrylbiphenyl Disulfonate, tetrabutyl Ethylidinebisphenol, F&DC Yellow 3, Fragrance.

Tide Stain Release Powder: Sodium percarbonate, sodium sulfate, sodium carbonate, sodium aluminosilicate, nonanoyloxy benzene sulfonate, sodium polyacrylate, water, sodium alkylbenzenesulfonate, DTPA, polyethylene glycol, sodium palmitate, amylase, protease, modified starch, FD&C Blue 1, fragrance.

Tide Stain Release, Pre Treater Spray: Water, Alkyl Ethoxylate, MEA Borate, Linear Alkylbenzenesulfonate, Propylene Glycol, Diquaternium Ethoxysulfate, Calcium Chlorideenzyme, Protease, Ethanolamine, Benzoisothiazolinone, Amylase, Sodium Citrate, Sodium Hydroxide, Fragrance.

Tide to Go Stain Eraser: Water, Alkyl Amine Oxide, Dipropylene Glycol Phenyl Ether, Hydrogen Peroxide, Citric Acid, Ethylene Diamine Disuccinic Acid Sodium salt, Sodium Alkyl Sulfate, Fragrance.

Tide boost with Oxi: Sodium bicarbonate, sodium carbonate, sodium percarbonate, alcohol ethoxylate, sodium chloride, maleic/acrylic copolymer, nonanoyloxy benzene sulfonate, sodium sulfate, colorant, diethylenetriamine pentaacetate sodium salt, hydrated aluminosilicate (zeolite), polyethylene glycol, sodium alkylbenzene sulfonate, sodium palmitate, starch, water, fragrance.

Tide Stain Release boost Duo Pac: Polyvinyl Alcoholpouch film, wherein there is packed a liquid part and a powder part: Liquid Ingredients: Dipropylene Glycol, diquaternium Ethoxysulfate, Water, Glycerin, Liquitint™ Orange, Powder Ingredients: sodium percarbonate, nonanoyloxy benzene sulfonate, sodium carbonate, sodium sulfate, sodium aluminosilicate, sodium polyacrylate, sodium alkylbenzenesulfonate, maleic/acrylic copolymer, water, amylase, polyethylene glycol, sodium palmitate, modified starch, protease, glycerine, DTPA, fragrance.

Tide Ultra Stain Release: Water, sodium alcoholethoxy sulfate, linear alkyl benzene sulfonate, sodium/MEA salts, MEA citrate, propylene glycol, polyethyleneimine ethoxylate, ethanol, diethylene glycol, polyethyleneimine propoxyethoxylate, sodium fatty acids, protease, borax, sodium cumene sulfonate, DTPA, fragrance, amylase, disodium diaminostilbene disulfonate, calcium formate, sodium formate, gluconase, dimethicone, Liquitint™ Blue, mannanase.

Ultra Tide with a Touch of Downy® Powdered Detergent, April Fresh/Clean Breeze/April Essence: Sodium Carbonate, Sodium Aluminosilicate, Sodium Sulfate, Linear Alkylbenzene Sulfonate, Bentonite, Water, Sodium Percarbonate, Sodium Polyacrylate, Silicate, Alkyl Sulfate, Nonanoyloxybenzenesulfonate, DTPA, Polyethylene Glycol 4000, Silicone, Ethoxylate, fragrance, Polyethylene Oxide, Palmitic Acid, Disodium Diaminostilbene Disulfonate, Protease, Liquitint™ Red, FD&C Blue 1, Cellulase.

Ultra Tide with a Touch of Downy Clean Breeze: Water, sodium alcoholethoxy sulfate, MEA citrate, linear alkyl benzene sulfonate: sodium/MEA salts, propylene glycol, polyethyleneimine ethoxylate, ethanol, diethylene glycol, polyethyleneimine, propoxyethoxylate, diquaternium ethoxysulfate, alcohol sulfate, dimethicone, fragrance, borax, sodium fatty acids, DTPA, protease, sodium bisulfite, disodium diaminostilbene disulfonate, amylase, gluconase, castor oil, calcium formate, MEA, styrene acrylate copolymer, sodium formate, Liquitint™ Blue.

Ultra Tide with Downy Sun Blossom: Water, sodium alcoholethoxy sulfate, MEA citrate, linear alkyl benzene sulfonate: sodium/MEA salts, propylene glycol, ethanol, diethylene glycol, polyethyleneimine propoxyethoxylate, polyethyleneimine ethoxylate, alcohol sulfate, dimethicone, fragrance, borax, sodium fatty acids, DTPA, protease, sodium bisulfite, disodium diaminostilbene disulfonate, amylase, castor oil, calcium formate, MEA, styrene acrylate copolymer, propanaminium propanamide, gluconase, sodium formate, Liquitint™ Blue.

Ultra Tide with Downy April Fresh/Sweet Dreams: Water, sodium alcoholethoxy sulfate, MEA citrate, linear alkyl benzene sulfonate: sodium/MEA salts, propylene glycol, polyethyleneimine ethoxylate, ethanol, diethylene glycol, polyethyleneimin propoxyethoxylate, diquaternium ethoxysulfate, alcohol sulfate, dimethicone, fragrance, borax, sodium fatty acids, DTPA, protease, sodium bisulfite, disodium diaminostilbene disulfonate, amylase, gluconase,

castor oil, calcium formate, MEA, styrene acrylate copolymer, propanaminium propanamide, sodium formate, Liquitint™ Blue.

Ultra Tide Free Powdered Detergent: Sodium Carbonate, Sodium Aluminosilicate, Alkyl Sulfate, Sodium Sulfate, Linear Alkylbenzene Sulfonate, Water, Sodium polyacrylate, Silicate, Ethoxylate, Sodium percarbonate, Polyethylene Glycol 4000, Protease, Disodium Diaminostilbene Disulfonate, Silicone, Cellulase.

Ultra Tide Powdered Detergent, Clean Breeze/Spring Lavender/mountain Spring: Sodium Carbonate, Sodium Aluminosilicate, Sodium Sulfate, Linear Alkylbenzene Sulfonate, Alkyl Sulfate, Sodium Percarbonate, Water, Sodium Polyacrylate, Silicate, Nonanoyloxybenzenesulfonate, Ethoxylate, Polyethylene Glycol 4000, Fragrance, DTPA, Disodium Diaminostilbene Disulfonate, Palmitic Acid, Protease, Silicone, Cellulase.

Ultra Tide HE (high Efficiency) Pwdered Detergent, Clean Breeze: Sodium Carbonate, Sodium Aluminosilicate, Sodium Sulfate, Linear Alkylbenzene Sulfonate, Water,

Nonanoyloxybenzenesulfonate, Alkyl Sulfate, Sodium Polyacrylate, Silicate, Sodium Percarbonate, Ethoxylate, Polyethylene Glycol 4000, Fragrance, DTPA, Palmitic Acid, Disodium Diaminostilbene Disulfonate, Protease, Silicone, Cellulase.

Ultra Tide Coldwater Powdered Detergent, Fresh Scent: Sodium Carbonate, Sodium Aluminosilicate, Sodium Sulfate, Sodium Percarbonate, Alkyl Sulfate, Linear Alkylbenzene Sulfonate, Water, Nonanoyloxybenzenesulfonate, Sodium Polyacrylate, Silicate, Ethoxylate, Polyethylene Glycol 4000, DTPA, Fragrance, Natalase, Palmitic Acid, Protease, Disodium, Diaminostilbene Disulfonate, FD&C Blue 1, Silicone, Cellulase, Alkyl Ether Sulfate.

Ultra Tide with bleach Powdered Detergent, Clean Breeze: Sodium Carbonate, Sodium Aluminosilicate, Sodium Sulfate, Linear Alkylbenzene Sulfonate, Sodium Percarbonate, Nonanoyloxybenzenesulfonate, Alkyl Sulfate, Water, Silicate, Sodium Polyacrylate, Ethoxylate, Polyethylene Glycol 4000, Fragrance, DTPA, Palmitic Acid, Protease, Disodium Diaminostilbene Disulfonate, Silicone, FD&C Blue 1, Cellulase, Alkyl Ether Sulfate.

Ultra Tide with Febreeze Freshness™ Powdered Detergent, Spring Renewal: Sodium Carbonate, Sodium Aluminosilicate, Sodium Sulfate, Linear Alkylbenzene Sulfonate, Sodium Percarbonate, Alkyl Sulfate, Water, Sodium Polyacrylate, Silicate, Nonanoyloxybenzenesulfonate, Ethoxylate, Polyethylene Glycol 4000, DTPA, Fragrance, Cellulase, Protease, Disodium Diaminostilbene Disulfonate, Silicone, FD&C Blue 1.

Liquid Tide Plus with Febreeze Freshness—Sport HE Active Fresh: Water, Sodium alcoholethoxy sulfate, MEA citrate, linear alkylbenzene sulfonate, sodium salt, linear alkylbenzene sulfonate: MEA salt, alcohol ethoxylate, sodium fatty acids, propylene glycol, diethylene glycol, polyethyleneimine ethoxylate propoxylate, diquaternium ethoxysulfate, Ethanol, sodium cumene sulfonate, borax, fragrance, DTPA, Sodium bisulfate, disodium diaminostilbene disulfonate, Mannanase, cellulase, amylase, sodium formate, calcium formate, Lauramine oxide, Liquitint™ Blue, Dimethicone/polydimethyl silicone.

Tide Plus Febreeze Freshness Spring & Renewal: Water, sodium alcoholethoxy sulfate, linear alkyl benzene sulfonate: sodium/MEA salts, MEA citrate, propylene glycol, polyethyleneimine ethoxylate, fragrance, ethanol, diethylene glycol, polyethyleneimine propoxyethoxylate, protease, alcohol sulfate, borax, sodium fatty acids, DTPA, disodium diaminostilbene disulfonate, MEA, mannanase, gluconase, sodium formate, dimethicone, Liquitint™ Blue, tetramine.

Liquid Tide Plus with Febreeze Freshness, Sport HE Victory Fresh: Water, Sodium alcoholethoxy sulfate, MEA citrate, linear alkylbenzene sulfonate, sodium salt, linear alkylbenzene sulfonate: MEA salt, alcohol ethoxylate, sodium fatty acids, propylene glycol, diethylene glycol, polyethyleneimine ethoxylate propoxylate, diquaternium ethoxysulfate, ethanol, sodium cumene sulfonate, borax, fragrance, DTPA, Sodium bisulfate, disodium diaminostilbene disulfonate, Mannanase, cellulase, amylase, sodium formate, calcium formate, Lauramine oxide, Liquitint™ Blue, Dimethicone/polydimethyl silicone.

Tide Vivid White+Bright Powder, Original: Sodium Carbonate, Sodium Aluminosilicate, Sodium Sulfate, Linear Alkylbenzene Sulfonate, Sodium Percarbonate, Nonanoyloxybenzenesulfonate, Alkyl Sulfate, Water, Silicate, Sodium Polyacrylate

### Ethoxylate, Polyethylene Glycol 4000, Fragrance, DTPA, Palmitic Acid, Protease, Disodium Diaminostilbene Disulfonate, Silicone, FD&C Blue 1, Cellulase, Alkyl Ether Sulfate.

Hey Sport Tex Wash Detergent: Aqua, dodecylbenzenesulfonsaure, laureth-11, peg-75 lanolin, propylene glycol, alcohol denat., potassium soyate, potassium hydroxide, disodium cocoamphodiacetate, ethylendiamine triacetate cocosalkyl acetamide, partum, zinc ricinoleate, sodium chloride, benzisothiazolinone, methylisothiazolinone, ci 16255, benzyl alcohol.

The products named Tide, Ariel, Gain and Fairy are commercially available products supplied by Procter & Gamble. The products named Persil are commercially available products supplied by Unilever and Henkel. The products named Hey Sport are commercially available products supplied by Hey Sport.

TABLE 1

| Ingredient | Amount (in wt %) |
|---|---|
| Anionic detersive surfactant (such as | from 8% to 15% |
| alkyl benzene sulphonate, alkyl | |
| ethoxylated sulphate and mixtures | |
| Non-ionic detersive surfactant | from 0.5% to 4% |
| (such as alkyl ethoxylated alcohol) | |
| Cationic detersive surfactant (such | from 0 to 4% |
| as quaternary ammonium compounds) | |
| Other detersive surfactant (such as | from 0% to 4% |
| zwiterionic detersive surfactants, | |
| amphoteric surfactants and mixtures | |
| thereof) | |
| Carboxylate polymer (such as | from 1% to 4% |
| co-polymers of maleic acid and | |
| acrylic acid) | |
| Polyethylene glycol polymer (such as | from 0.5% to 4% |
| a polyethylene glycol polymer comprising | |
| poly vinyl acetate side chains) | |
| Polyester soil release polymer (such as | from 0.1 to 2% |
| Repel-o-tex from and/or Texcare | |
| polymers) | |
| Cellulosic polymer (such as | from 0.5% to 2% |
| carboxymethyl cellulose, methyl | |
| cellulose and combinations thereof) | |
| Other polymer (such as amine polymers, | from 0% to 4% |
| dye transfer inhibitor polymers, | |
| hexamethylenediamine derivative polymers, | |
| and mixtures thereof) | |
| Zeolite builder and phosphate builder | from 0% to 4 wt % |
| (such as zeolite 4A and/or | |
| sodium tripolyphosphate) | |
| Other builder | from 0% to 3% |
| (such as sodium citrate and/or citric acid) | |
| Carbonate salt | from 15% to 30% |
| (such as sodium carbonate and/ | |
| or sodium bicarbonate) | |
| Silicate salt (such as sodium silicate) | from 0% to 10% |
| Filler (such as sodium sulphate | from 10% to 40% |
| and/or bio-fillers) | |
| Source of available oxygen | from 10% to 20% |
| (such as sodium percarbonate) | |
| Bleach activator | from 2% to 8% |
| (such as tetraacetylethylene | |
| diamine (TAED) and/or | |
| nonanoyloxybenzenesulphonate (NOBS) | |
| Bleach catalyst | from 0% to 0.1% |
| (such as oxaziridinium- | |
| based bleach catalyst and/or | |
| transition metal bleach catalyst) | |
| Other bleach | from 0% to 10% |
| (such as reducing bleach | |
| and/or pre-formed peracid) | |
| Chelant | from 0.2% to 1% |
| (such as ethylenediamine- | |
| N′N′-disuccinic acid (EDDS) and/or | |
| hydroxyethane diphosphonic acid(HEDP) | |
| Photobleach (such as zinc and/ | from 0% to 0.1% |
| or aluminium sulphonated phthalocyanine) | |
| Hueing agent | from 0% to 1% |
| (such as direct violet 99, | |
| acid red 52, acid blue 80, direct | |
| violet 9, solvent violet 13 | |
| and any combination thereof) | |
| Brightener | from 0.1% to 0.4% |
| (such as brightener 15 | |
| and/or brightener 49) | |
| Protease such as those mentioned under | from 0.1% to 0.4% |
| the heading “proteases” e.g. | |
| Savinase, Savinase Ultra, Ovozyme, | |
| Kannase, Liquanase, Polarzyme, | |
| Purafect, Properase, FN3, | |
| FN4 and any combination thereof) | |
| Amylase (such as Termamyl, | from 0.05% to 0.2% |
| Termamyl ultra Natalase, Optisize, | |
| Stainzyme, Stainzyme Plus, | |
| and any combination thereof) | |
| Cellulase | from 0.05% to 0.2% |
| (such as Carezyme and/or Celluclean) | |
| Lipase (such as Lipex, Lipolex, | from 0.2 to 1% |
| Lipoclean and any combination | |
| thereof) | |
| Other enzyme | from 0% to 2% |
| (such as xyloglucanase, | |
| cutinase, pectate lyase, | |
| mannanase, bleaching enzyme) | |
| Fabric softener | from 0% to 4% |
| (such as montmorillonite clay and/or | |
| polydimethylsiloxane (PDMS) | |
| Flocculant (such as polyethylene oxide) | from 0% to 1% |
| Suds suppressor | from 0% to 0.1% |
| (such as silicone and/or fatty acid) | |
| Perfume | from 0.1% to 1% |
| (such as perfume microcapsule, | |
| spray-on perfume, starch encapsulated | |
| perfume accords, perfume loaded zeolite, | |
| and any combination thereof) | |
| Aesthetics | from 0% to 1% |
| (such as coloured soap | |
| rings and/or coloured | |
| speckles/noodles) | |
| Miscellaneous | balance |

TABLE 2

| Ingredient | Amount |
|---|---|
| Carboxyl group-containing polymer (comprising from about 60% to | from about 0.5 wt % to about 1.5 wt % |
| about 70% by mass of an acrylic acid-based monomer (A); and from | |
| about 30% to about 40%) by mass of a sulfonic acid group-containing | |
| monomer (B); and wherein the average molecular weight is from | |
| about 23,000 to about 50,000 preferably in the range of from about | |
| 25,000 to about 38,000 as described in WO2014032269. | |
| Amylase (Stainzyme Plus(R), having an enzyme activity of 14 mg | from about 0.1 wt % to about 0.5 wt % |
| active enzyme/g) | |
| Anionic detersive surfactant (such as alkyl benzene sulphonate, alkyl | from about 8 wt % to about 15 wt % |
| ethoxylated sulphate and mixtures thereof) | |
| Non-ionic detersive surfactant (such as alkyl ethoxylated alcohol) | from about 0.5 wt % to 4 wt % |
| Cationic detersive surfactant (such as quaternary ammonium | from about 0 wt % to about 4 wt % |
| compounds) | |
| Other detersive surfactant (such as zwiterionic detersive surfactants, | from about 0 wt % to 4 wt % |
| amphoteric surfactants and mixtures thereof) | |
| Carboxylate polymer (such as co-polymers of maleic acid and acrylic | from about 1 wt % to about 4 wt % |
| acid) | |
| Polyethylene glycol polymer (such as a polyethylene glycol polymer | from about 0 wt % to about 4 wt % |
| comprising poly vinyl acetate side chains) | |
| Polyester soil release polymer (such as Repel-O-Tex(R) and/or | from about 0.1 wt % to about 2 wt % |
| Texcare(R) polymers) | |
| Cellulosic polymer (such as carboxymethyl cellulose, methyl cellulose | from about 0.5 wt % to about 2 wt % |
| and combinations thereof) | |
| Other polymer (such as amine polymers, dye transfer inhibitor | from about 0 wt % to about 4 wt % |
| polymers, hexamethylenediamine derivative polymers, and mixtures | |
| thereof) | |
| Zeolite builder and phosphate builder (such as zeolite 4A and/or | from about 0 wt % to about 4 wt % |
| sodium tripolyphosphate) | |
| Other builder (such as sodium citrate and/or citric acid) | from about 0 wt % to about 3 wt % |
| Carbonate salt (such as sodium carbonate and/or sodium | from about 15 t % to about 30 wt % |
| bicarbonate) | |
| Silicate salt (such as sodium silicate) | from about 0 wt % to about 10 wt % |
| Filler (such as sodium sulphate and/or bio-fillers) | from about 10 wt % to about 40 wt % |
| Source of available oxygen (such as sodium percarbonate) | from about 10 wt % to about 20 wt % |
| Bleach activator (such as tetraacetylethylene diamine (TAED) and/or | from about 2 wt % to about 8 wt % |
| nonanoyloxybenzenesulphonate (NOBS) | |
| Bleach catalyst (such as oxaziridinium-based bleach catalyst and/or | from about 0 wt % to about 0.1 wt % |
| transition metal bleach catalyst) | |
| Other bleach (such as reducing bleach and/or pre formed peracid) | from about 0 wt % to about 10 wt % |
| Chelant (such as ethylenediamine-N′N′-disuccinic acid (EDDS) and/or | from about 0.2 wt % to about 1 wt % |
| hydroxyethane diphosphonic acid (HEDP) | |
| Photobleach (such as zinc and/or aluminium sulphonated | from about 0 wt % to about 0.1 wt % |
| phthalocyanine) | |
| Hueing agent (such as direct violet 99, acid red 52, acid blue 80, direct | from about 0 wt % to about 0.5 wt % |
| violet 9, solvent violet 13 and any combination thereof) | |
| Brightener (such as brightener 15 and/or brightener 49) | from about 0.1 wt % to about 0.4 wt % |
| Protease such as those mentioned under the heading “proteases” e.g. | from about 0.1 wt % to about 1.5 wt % |
| Savinase, Coronase, Ovozyme, Kannase, Liquanase, Polarzyme, | |
| Purafect, Properase, FN3, FN4 and any combination thereof, typically | |
| having an enzyme activity of from about 20 mg to about 100 mg active | |
| enzyme/g) | |
| Amylase (such as Termamyl(R), Termamyl Ultra(R), Natalase(R), | from about 0.05 wt % to about 0.2 wt % |
| Optisize HT Plus(R), Powerase(R), Stainzyme(R) and any combination thereof, | |
| typically having an enzyme activity of from about | |
| 10 mg to about 50 mg active enzyme/g) | |
| Cellulase (such as Carezyme(R), Celluzyme(R) and/or Celluclean(R), | from about 0.05 wt % to 0.5 wt % |
| typically having an enzyme activity of about from 10 to 50 mg active | |
| enzyme/g) | |
| Lipase (such as Lipex(R), Lipolex(R), Lipoclean(R) and any | from about 0.2 wt % to about 1 wt % |
| combination thereof, typically having an enzyme activity of from about | |
| 10 mg to about 50 mg active enzyme/g) | |
| Other enzyme (such as xyloglucanase (e.g., Whitezyme(R)), cutinase, | from 0 wt % to 2 wt % |
| pectate lyase, mannanase, bleaching enzyme, typically having an | |
| enzyme activity of from about 10 mg to about 50 mg active enzyme/g) | |
| Fabric softener (such as montmorillonite clay and/or | from 0 wt % to 15 wt % |
| polydimethylsiloxane (PDMS)) | |
| Flocculant (such as polyethylene oxide) | from 0 wt % to 1 wt % |
| Suds suppressor (such as silicone and/or fatty acid) | from 0 wt % to 0.1 wt % |
| Perfume (such as perfume microcapsule, spray-on perfume, starch | from 0.1 wt % to 1 wt % |
| encapsulated perfume accords, perfume loaded zeolite, and any | |
| combination thereof) | |
| Aesthetics (such as colored soap rings and/or colored | from 0 wt % to 1 wt % |
| speckles/noodles) | |
| Miscellaneous | Balance |

All enzyme levels expressed as rug active enzyme protein per 100 g detergent composition. Surfactant ingredients can be obtained from BASF, Ludwigshafen, Germany (Lutensol®); Shell Chemicals, London, UK; Stepan, Northfield, Ill., USA; Huntsman, Huntsman, Salt Lake City, Utah, USA; Clariant, Sulzbach, Germany (Praepagen®).

Sodium tripolyphosphate can be obtained from Rhodia, Paris, France.

Zeolite can be obtained from Industrial Zeolite (UK) Ltd, Grays, Essex, UK.

Citric acid and sodium citrate can be obtained from Jungbunzlauer, Basel, Switzerland. NOBS is sodium nonanoyloxybenzenesulfonate, supplied by Eastman, Batesville, Ark., USA.

TAED is tetraacetylethylenediamine, supplied under the Peractive® brand name by Clariant GmbH, Sulzbach, Germany.

Sodium carbonate and sodium bicarbonate can be obtained from Solvay, Brussels, Belgium.

Polyacrylate, polyacrylate/maleate copolymers can be obtained from BASF, Ludwigshafen, Germany.

Repel-O-Tex® can be obtained from Rhodia, Paris, France.

Texcare® can be obtained from Clariant, Sulzbach, Germany. Sodium percarbonate and sodium carbonate can be obtained from Solvay, Houston, Tex., USA.

Na salt of Ethylenediamine-N,N′-disuccinic acid, (S,S) isomer (EDDS) was supplied by Octel, Ellesmere Port, UK.

Hydroxy ethane di phosphonate (HEDP) was supplied by Dow Chemical, Midland, Mich., USA. Enzymes Savinase®, Savinase® Ultra, Stainzyme® Plus, Lipex®, Lipolex®, Lipoclean®, Celluclean®, Carezyme®, Natalase®, Stainzyme®, Stainzyme® Plus, Termamyl®, Termamyl® ultra, and Mannaway® can be obtained from Novozymes, Bagsvaerd, Denmark.

Enzymes Purafect®, FN3 and FN4 can be obtained from DuPont International Inc., Palo Alto, Calif., US. Direct violet 9 and 99 can be obtained from BASF DE, Ludwigshafen, Germany. Solvent violet 13 can be obtained from Ningbo Lixing Chemical Co., Ltd. Ningbo, Zhejiang, China. Brighteners can be obtained from Ciba Specialty Chemicals, Basel, Switzerland. All percentages and ratios are calculated by weight unless otherwise indicated. All percentages and ratios are calculated based on the total composition unless otherwise indicated. It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

### Wash Assays

### Launder-O-Meter (LOM) Model Wash System

The Launder-O-Meter (LOM) is a medium scale model wash system that can be applied to test up to 20 different wash conditions simultaneously. A LOM is basically a large temperature controlled water bath with 20 closed metal beakers rotating inside it. Each beaker constitutes one small washing machine and during an experiment, each will contain a solution of a specific detergent/enzyme system to be tested along with the soiled and unsoiled fabrics it is tested on. Mechanical stress is achieved by the beakers being rotated in the water bath and by including metal balls in the beaker.

The LOM model wash system is mainly used in medium scale testing of detergents and enzymes at European wash conditions. In a LOM experiment, factors such as the ballast to soil ratio and the fabric to wash liquor ratio can be varied. Therefore, the LOM provides the link between small scale experiments, such as AMSA and mini-wash, and the more time consuming full scale experiments in front loader washing machines.

### Mini Launder-O-Meter (MiniLOM) Model Wash System

MiniLOM is a modified mini wash system of the Launder-O-Meter (LOM), which is a medium scale model wash system that can be applied to test up to 20 different wash conditions simultaneously. A LOM or is basically a large temperature controlled water bath with 20 closed metal beakers rotating inside it. Each beaker constitutes one small washing machine and during an experiment, each will contain a solution of a specific detergent/enzyme system to be tested along with the soiled and unsoiled fabrics it is tested on. Mechanical stress is achieved by the beakers being rotated in the water bath and by including metal balls in the beaker.

The LOM model wash system is mainly used in medium scale testing of detergents and enzymes at European wash conditions. In a LOM experiment, factors such as the ballast to soil ratio and the fabric to wash liquor ratio can be varied. Therefore, the LOM provides the link between small scale experiments, such as AMSA and mini-wash, and the more time consuming full scale experiments in front loader washing machines.

In miniLOM, washes are performed in 50 ml test tubes placed in Stuart rotator.

### Terg-O-Tometer (TOM) Wash Assay

The Terg-O-tometer (TOM) is a medium scale model wash system that can be applied to test 12 different wash conditions simultaneously. A TOM is basically a large temperature controlled water bath with up to 12 open metal beakers submerged into it. Each beaker constitutes one small top loader style washing machine and during an experiment, each of them will contain a solution of a specific detergent/enzyme system and the soiled and unsoiled fabrics its performance is tested on. Mechanical stress is achieved by a rotating stirring arm, which stirs the liquid within each beaker. Because the TOM beakers have no lid, it is possible to withdraw samples during a TOM experiment and assay for information on-line during wash.

The TOM model wash system is mainly used in medium scale testing of detergents and enzymes at US or LA/AP wash conditions, as well as for EU conditions. In a TOM experiment, factors such as the ballast to soil ratio and the fabric to wash liquor ratio can be varied. Therefore, the TOM provides the link between small scale experiments and the more time consuming full scale experiments in top loader washing machines.

### Production of CBM

Expression constructs were constructed by preparing a shuttle plasmid comprising the nucleotide sequence encoding the CBM in operation connection with an Aspergillus promoter, signal sequence and Kex cleavage site and terminator, and further comprising an amdS gene for amdS selection in Aspergillus. The promoter used for the CBM production is further described in WO2003/008575. The correctness of the constructs was confirmed by sequencing.

Aspergillus transformation: An Aspergillus oryzae laboratory strain was transformed with the expression constructs and grown under inductive conditions for expression of the CBM.

Recovery of CBM: After growing the transformed Aspergillus, the CBM was purified from the supernatant using standard chromatographic methods.

### Example 1

### Preparation of CBMs

Three CBMs, belonging to the CBM1 family, were prepared as described under Methods and Materials

CBM1-1 was derived from Fusarium longipes GH10 polypeptide and was encoded by the nucleotide sequence:

(SEQ ID NO: 1)
cagtcccccatctggggacagtgtggtggaaacggatggactggtgcaac
aacatgtcagtccggactcaagtgtgagaaagtgaacgattggtactacc
agtgtgtcccctaa

and had the amino acid sequence:

(SEQ ID NO: 2)
QSPIWGQCGGNGWTGATTCQSGLKCEKVNDWYYQCVP

CBM1-2 was derived from Fusarium longipes GH6 polypeptide and was encoded by the nucleotide sequence:

(SEQ ID NO: 3)
gcaccggtcgaagaacgacagtcgtgttcgaacggagtctgggcacagtg
tggtggtcagaactggtcgggtacaccctgttgtacatccggcaacacat
gtgtcaaaatcaacgacttctactcgcagtgtcagcctggctaa

and had the amino acid sequence:

(SEQ ID NO: 4)
APVEERQSCSNGVWAQCGGQNWSGTPCCTSGNTCVKINDFYSQCQPG

CBM1-3 was derived from Aspergillus clavatus carbohydrate esterase CE1 polypeptide and was encoded by the nucleotide sequence:

(SEQ ID NO: 5)
cagcagtccctctatggccagtgtggaggtaacggctggtccggacccac
agagtgtacagcaggagcatgttgtcaggtccagaacccgtggtattccc
agtgtctccctggcgattgttaa

and had the amino acid sequence:

(SEQ ID NO: 6)
QQSLYGQCGGNGWSGPTECTAGACCQVQNPWYSQCLPGDC

Additional CBMs of various CBM families were prepared. The overall cloning and transformation methods are the same as in the Materials and Methods section, but the genes encoding for the recombinant CBMs were codon-optimized for Aspergillus oryzae and synthesized by GeneArt.

The signal peptide sequence MKLSWLVAAALTAASVVSA (SEQ ID NO: 21) was used for secretion of the recombinant CBMs.

CBM79 was derived from Ruminococcus flavefaciens GH9 endoglucanase polypeptide and was encoded by the nucleotide sequence of SEQ ID NO: 7 and has the amino acid sequence:

(SEQ ID NO: 8)
DGYTIKPNKKVTYSALGEDERMIGFSYKDFGISSSEKITEVQVNI-
SANKNIGKYVGQFGTSTTDSANGYWAMGDEITQSISGNSGTITWKVPSDI
SSIIQTQYGGEIKFGVWWIDCDEFTIDSVVLK

CBM72 was derived from unidentified microorganism GH5 endoglucanase polypeptide and was encoded by the nucleotide sequence of SEQ ID NO: 9 and has the amino acid sequence:

(SEQ ID NO: 10)
GYKYPTADDFEIVYDISYNDEWSELFLFGSWDRTAVNLSGYKGIRVEMDK
AYGNKLQIKVYG-DKKSGTDFNEQYAPLSDTSASTTVDFDTSILGSTFWG
VTLOTNSGALTATLKEAKLIKADGTEEPASVTAAWGCTVTAKSTPKPTGI
HAIQLIKTEADGAIYNLQGQRVQNPQKGIYIQNGKKYVMK

CBM44 was derived from Hungateiclostridium thermocellum GH9 endoglucanase polypeptide and was encoded by the nucleotide sequence of SEQ ID NO: 11 and has the amino acid sequence:

(SEQ ID NO: 12)
GTLGGFTTSGTNATGVVVNTTEKAFKGERGLKWTVTSEGEGTAELKLDGG
TIVVPGTT-MTFRIWIPSGAPIAAIQPYIMPHTPDWSEVLWNSTWKGYTM
VKTDDWNEITLTLPEDVDPTWPQQMGIQVQTIDEGEFTIYVDAIDW

The produced protein contains 19,9% of protein with sequence of SEQ ID NO: 12 and 80,1% of protein having the mutation G134S.

CBM30 was derived from Clostridium cellulovorans GH9 endoglucanase polypeptide and was encoded by the nucleotide sequence of SEQ ID NO: 13 and has the amino acid sequence:

(SEQ ID NO: 14)
KLMDLEVFKSASITGWSGSAGGELEVASDSNLPIDTSATYNGLPSLRLNV
TKASAQWWS-SLLTLRGWCTQDLTQYLANGYLEFNVKGKVGGEDFQIGLQ
DQTHERAAGDSVTSVKSIKNYVNISTNWQHVKIPLKDIMGPSTGFDPTTA
RCINIVKGSSEIFTAWINDLKITSTDNEK

A heterodimer comprising CBM17 and CBM28 was derived from Clostridium cellulovorans GH5 endoglucanase polypeptide and was encoded by the nucleotide sequence of SEQ ID NO: 15 and has the amino acid sequence:

(SEQ ID NO: 16)
LWDFNDGTKQGFGVNGDSPVEDVVIENEAGALKLSGLDASNDVSEGNYWA
NARLSADG-WGKSVDILGAEKLTMDVIVDEPTTVSIAAIPQGPSANWVNP
NRAIKVEPTNFVPLGDKFKAELTITSADSPSLEAIAMHAENNNINNIILF
VGTEGADVIYLDNIKVIG-TEVEIPVVHDPKGEAVLPSVFEDGTRQGWDW
AGESGVKTALTIEEANGSNALSWEFGYPEVKPSDNWATAPRLDFWKSDLV
RGENDYVTFDFYLDPVRATEGAMNINLVFQPPTNGYWVQAP-KTYTINFD
ELEEANQVNGLYHYEVKINVRDITNIQDDTLLRNMMIIFADVESDFAGRV
FVDNVRFEGAATTE

The produced protein also includes protein having the mutation V174M.

(SEQ ID NO: 17)
LWDFNDGTKQGFGVNGDSPVEDVVIENEAGALKLSGLDASNDVSEGNYWA
NARLSADG-WGKSVDILGAEKLTMDVIVDEPTTVSIAAIPQGPSANWVNP
NRAIKVEPTNFVPLGDKFKAELTITSADSPSLEAIAMHAENNNINNIILF
VGTEGADVIYLDNIKVI
and
(SEQ ID NO: 18)
GTEVEIPVVHDPKGEAVLPSVFEDGTRQGWDWAGESGVKTALTIEEANGS
NAL-SWEFGYPEVKPSDNWATAPRLDFWKSDLVRGENDYVTFDFYLDPVR
ATEGAMNINLVFQPPTNGYWVQAPKTYTINFDELEEANQVNGLYHYEVKI
NVRDITNIQDDTLLRNMMIIFAD-VESDFAGRVFVDNVRFEGAATTE

correspond to the CBM17 and CBM28 portions, respectively.

CBM4 was derived from Cellulomonas fimi GH9 endoglucanase polypeptide and was encoded by the nucleotide sequence of SEQ ID NO: 19 and has the amino acid sequence:

(SEQ ID NO: 20)
ASPIGEGTFDDGPEGWVAYGTDGPLDTSTGALCVAVPAGSAQYGVGVVLN
GVAIEEGTTYTL-RYTATASTDVTVRALVGQNGAPYGTVLDTSPALTSEP
RQVTETFTASATYPATPAADDPEGQIAFQLGGFSADAWTFCLDDVALDSE
VELLP

### Example 2

CBM Anti-Crease Properties with Mixed Soil from Soil Ballast Evaluated on Cotton T-Shirts

Blue T-shirts for children produced in Bangladesh were purchased from ZARA, China. T-shirts were used as tracers for wrinkle count. 4 pieces of soil-ballast (SBL-CFT) in size 40×20 cm² equalizing 8 g soil were added to each European front loader Full Scale Wash (FSW) machine. For FSW was employed Miele Softtronic W5841 washing machine (Program: Cottons; Additional program: Short; Temperature: 30° C.; Centrifuge: 1600 rpm; Ballast: 600-700 g 100% cotton T-shirts). A commercial detergent composition, Ariel Color & Style, was dosed 5 g/L. Three carbon-binding module prepared in Example 1, dosed 0.5 ppm were added to individual washing machines and laundered as described. 4 independent replica of each FSW were conducted. From each machine T-shirts were line-dried for 24 h at room temperature. Fabric pieces were evaluated by scoring according to the Standard AATCC Three-Dimensional Smoothness Appearance Replicas by a panel consisting of 7 panelists (the panel set-up was as close to AATCC method 124 as possible). Panelists were asked to compare each swatch with the AATCC smoothness standards ranking from SA value=1 (very wrinkled standard) to SA value 5=(totally smooth standard). After evaluation, average and standard error across the panel scores was calculated for each condition.

Textile evaluated by AATCC Smoothness
standards Average SA-value according
to AATCC +/− stE on average

| Protein | −CBM | +CBM |
|---|---|---|
| CBM1-1 | 1.8 +/− 0.4 | 3.1 +/− 0.3 |
| SEQ ID NO 2 | | |
| (0.5 ppm) | | |
| CBM1-2 | 1.8 +/− 0.4 | 2.5 +/− 0.5 |
| SEQ ID NO 4 | | |
| (0.5 ppm) | | |
| CBM1-3 | 1.8 +/− 0.4 | 2.5 +/− 0.2 |
| SEQ ID NO 6 | | |
| (0.5 ppm) | | |

Values specify the average SA value rank given by the panel according to the AATCC smoothness standards +/− StE.

### Example 3

A Mixture of Two CBM Classes Having Anti-Crease Properties with Mixed Soil from Soil Ballast Evaluated on Cotton T-Shirts

Pink T-shirts (100% cotton) for girls produced in India were purchased from Decathlon, France. 4 T-shirts per machine were used as tracers for wrinkle count. 4 pieces of soil-ballast (SBL-CFT) in size 40×20 cm² equalizing 8 g soil were added to each European front loader Full Scale Wash (FSW) machine. Washes were done using Miele Softtronic W5841 washing machine (Program: Cottons; Additional program: Short; Temperature: 30° C.; Centrifuge: 800 rpm; Ballast: 600-700 g 100% cotton T-shirts. Model Detergent B was dosed 3.3 g/L. A mixture of two carbon-binding modules (SEQ ID NO: 17 and SEQ ID NO: 18, CBM17 and CBM28, respectively) were dosed as below. From each machine T-shirts were line-dried for 24 h at room temperature. Fabric pieces were evaluated by scoring according to the Standard AATCC Three-Dimensional Smoothness Appearance Replicas by a panel consisting of 4 trained panelists (the panel set-up was as close to AATCC method 124 as possible). Panelists were asked to compare each swatch with the AATCC smoothness standards ranking from SA value=1 (very wrinkled standard) to SA value 5=(totally smooth standard). After evaluation, average and standard error across the panel scores was calculated for each condition.

| | | | | | Textile evaluated by AATCC | |
|---|---|---|---|---|---|---|
| | | | | | Smoothness standards | |
| | | | Soiled/clean | | Average SA-value according | |
| | FSW wash | | Textile in | Drying | to AATCC +/− stE on average | |
| Protein | conditions | Detergent | the wash | regime | −CBM-mix | +CBM-mix |
| CBM17 + CBM28 | Cottons wash; | Liquid Model B | 4 T-shirts | Line | 1.5 +/− 0.1 | 2.2 +/− 0.1 |
| SEQ ID NO: 17 and | Additional program: | (3.3 g/L) | Soiled SBL-CFT | dry | | |
| SEQ ID NO: 18 | Short; | | Clean 100% | | | |
| (0.25 total | Temperature: | | cotton T-shirt | | | |
| ppm of mix) | 30° C.; | | ballast. | | | |
| | Centrifuge: | | | | | |
| | 800 rpm | | | | | |
| CBM17 + CBM28 | Cottons wash; | Liquid Model B | 4 T-shirts | Line | 1.5 +/− 0.1 | 2.3 +/− 0.3 |
| SEQ ID NO: 17 and | Additional program: | (3.3 g/L) | Soiled SBL-CFT | dry | | |
| SEQ ID NO: 18 | Short; | | Clean 100% | | | |
| (0.5 ppm of mix) | Temperature: | | cotton T-shirt | | | |
| | 30° C.; | | ballast. | | | |
| | Centrifuge: | | | | | |
| | 800 rpm | | | | | |
| CBM17 + CBM28 | Cottons wash; | Liquid | 4 T-shirts | Line | 1.5 +/− 0.1 | 2.5 +/− 0.2 |
| SEQ ID NO: 17 and | Additional program: | Model B | Soiled SBL-CFT | dry | | |
| SEQ ID NO: 18 | Short; | (3.3 g/L) | Clean 100% | | | |
| (2 ppm of mix) | Temperature: | | cotton T-shirt | | | |
| | 30° C.; | | ballast. | | | |
| | Centrifuge: | | | | | |
| | 800 rpm | | | | | |

Values specify the average SA value rank given by the panel according to the AATCC smooth-ness standards +/− StE.

### Example 4

A CBM44 Class CBM Having Anti-Crease Properties with Mixed Soil from Soil Ballast Evaluated on Cotton T-Shirts

Pink T-shirts (100% cotton) for girls produced in India were purchased from Decathlon, Germany. 4 T-shirts per machine were used as tracers for wrinkle count. 4 pieces of soil-ballast (SBL-CFT) in size 40×20 cm² equalizing 8 g soil were added to each European front loader Full Scale Wash (FSW) machine. Washes were done using Miele Softtronic W5841 washing machine (Program: Cottons; Additional program: Short; Temperature: 30° C.; Centrifuge: 800 rpm; Ballast: 4 kg 100% cotton T-shirts). Model Detergent B was dosed 3.3 g/L. A CBM44 carbon-binding modules was tested (SEQ ID NO: 12) dosed 0.25 ppm; 0.5 ppm and 2 ppm respectively. From each machine T-shirts were line-dried for 24 h at room temperature. Fabric pieces were evaluated by scoring according to the Standard AATCC Three-Dimensional Smoothness Appearance Replicas by a panel consisting of 4 trained panelists (the panel set-up was as close to AATCC method 124 as possible). Panelists were asked to compare each swatch with the AATCC smoothness standards ranking from SA value=1 (very wrinkled standard) to SA value 5=(totally smooth standard). After evaluation, average and standard error across the panel scores was calculated for each condition.

| | | | | | Textile evaluated by AATCC | |
|---|---|---|---|---|---|---|
| | | | | | Smoothness standards | |
| | | | Soiled/clean | | Average SA-value according | |
| | FSW wash | | Textile in | Drying | to AATCC +/− stE on average | |
| Protein | conditions | Detergent | the wash | regime | −CBM44 | +CBM44 |
| CBM44 | Cottons wash; | Liquid Model B | 4 T-shirts | Line | 1.5 +/− 0.1 | 2.2 +/− 0.2 |
| SEQ ID NO: 12 | Additional program: | (3.3 g/L) | Soiled SBL-CFT | dry | | |
| (0.25 ppm) | Short; | | Clean 100% | | | |
| | Temperature: | | cotton T-shirt | | | |
| | 30° C.; | | and shirt ballast. | | | |
| | Centrifuge: | | | | | |
| | 800 rpm | | | | | |
| CBM44 | Cottons wash; | Liquid Model B | 4 T-shirts | Line | 1.5 +/− 0.1 | 2.3 +/− 0.3 |
| SEQ ID NO: 12 | Additional program: | (3.3 g/L) | Soiled SBL-CFT | dry | | |
| (0.5 ppm) | Short; | | Clean 100% | | | |
| | Temperature: | | cotton T-shirt | | | |
| | 30° C.; | | and shirt ballast. | | | |
| | Centrifuge: | | | | | |
| | 800 rpm | | | | | |
| CBM44 | Cottons wash; | Liquid Model B | 4 T-shirts | Line | 1.5 +/− 0.1 | 2.3 +/− 0.2 |
| SEQ ID NO: 12 | Additional program: | (3.3 g/L) | Soiled SBL-CFT | dry | | |
| (2 ppm) | Short; | | Clean 100% | | | |
| | Temperature: | | cotton T-shirt | | | |
| | 30° C.; | | and shirt ballast. | | | |
| | Centrifuge: | | | | | |
| | 800 rpm | | | | | |

Values specify the average SA value rank given by the panel according to the AATCC smooth-ness standards +/− StE

### Example 5

A Mixture of Three CBM1 Monomers Giving Shape Retention Properties within First Wash with Mixed Soil from Soil Ballast Evaluated on Cotton T-Shirts

Pink T-shirts (100% cotton) for girls produced in India were purchased from Decathlon, France. 4 T-shirts per machine were used as tracers for shape retention assessment by a panel. 4 pieces of soil-ballast (SBL-CFT) in size 40×20 cm² equalizing 8 g soil were added to each European front loader Full Scale Wash (FSW) machine. Washes were done using Miele Softtronic W5841 washing machine (Program: Cottons; Additional program: Short; Temperature: 30° C.; Centrifuge: 800 rpm; Ballast: 600-700 g 100% cotton T-shirts. Model Detergent B was dosed 3.3 g/L. A mixture of three carbon-binding modules (monomeric CBM1-1, CBM1-2 and CBM1-3, (SEQ ID NO:s 2; 4; 6 respectively)) were tested with total dose as below. From each machine T-shirts were line-dried for 24 h at room temperature. Sets of T-shirts from 3 individual trials were scored during the same panel scoring. Fabric pieces were evaluated by preference scoring by a panel consisting of 24 non-trained panelists (randomized preference test between pairs of each treatment). Panelists were asked to point out the preferred shape according to original shape. After evaluation, percentage of each preference was calculated.

| | | | Soiled/clean | | | |
|---|---|---|---|---|---|---|
| | FSW wash | | Textile in | Drying | Preference test % preferred | |
| Protein | conditions | Detergent | the wash | regime | −CBM1-mixture | +CBM1-mixture |
| CBM1-1 | Cottons wash; | Model B, | 4 T-shirts | Line | 16 | 84 |
| SEQ ID NO 2 | Additional program: | 3.3 g/L | Soiled SBL-CFT | dry | | |
| CBM1-2 | Short; | | Clean 100% | | | |
| SEQ ID NO 4, | Temperature: | | cotton T-shirt | | | |
| CBM1-3 | 30° C.; | | ballast. | | | |
| SEQ ID NO 6 | Centrifuge: | | | | | |
| (total dose | 800 rpm | | | | | |
| 0.5 ppm) | | | | | | |
| CBM1-1 | Cottons wash; | Model B, | 4 T-shirts | Line | 21 | 79 |
| SEQ ID NO 2 | Additional program: | 3.3 g/L | Soiled SBL-CFT | dry | | |
| CBM1-2 | Short; | | Clean 100% | | | |
| SEQ ID NO 4, | Temperature: | | cotton T-shirt | | | |
| CBM1-3 | 30° C.; | | ballast. | | | |
| SEQ ID NO 6 | Centrifuge: | | | | | |
| (total dose | 800 rpm | | | | | |
| 2 ppm) | | | | | | |

### Example 6

CBM4 and CBM72—Two Different CBM Classes Anti-Crease Properties with Mixed Soil from Soil Ballast Evaluated on Cotton T-Shirts

Pink T-shirts for children produced in Bangladesh were purchased from Decathlon, F. T-shirts were used as tracers for wrinkle count. 4 pieces of soil-ballast (SBL-CFT) in size 40×20 cm² equalizing 8 g soil were added to each European front loader Full Scale Wash (FSW) machine. Washes were done using Miele Softtronic W5841 washing machine (Program: Cottons; Additional program: Short; Temperature: 30° C.; Centrifuge: 1600 rpm; Ballast: 600-700 g 100% cotton T-shirts). Ariel Color & Style was dosed 5 g/L. Two representatives of carbon-binding module from two different CBM-classes (CBM4 and CBM72, SEQ ID NO: 18 and 10, respectively) were dosed 0.5 ppm. 4 independent replica of each FSW were conducted. From each machine T-shirts were line-dried for 24 h at room temperature. Fabric pieces were evaluated by scoring according to the Standard AATCC Three-Dimensional Smoothness Appearance Replicas by a panel consisting of 3 panelists (the panel set-up was as close to AATCC method 124 as possible). Panelists were asked to compare each swatch with the AATCC smoothness standards ranking from SA value=1 (very wrinkled standard) to SA value 5=(totally smooth standard). After evaluation, average and standard error across the panel scores was calculated for each condition.

| | | | | | Textile evaluated by AATCC | |
|---|---|---|---|---|---|---|
| | | | | | Smoothness standards | |
| | | | Soiled/clean | | Average SA-value according to | |
| | FSW wash | | Textile in | Drying | AATCC +/− stE on average | |
| Protein | conditions | Detergent | the wash | regime | −CBM | +CBM |
| CBM4 | Cottons wash; | Liquid Ariel Color | Zara T-shirts | Line | 1.61 +/− 0.18 | 1.81 +/− 0.13 |
| SEQ ID NO: 20 | Additional program: | and Style | Soiled SBL-CFT | dry | | |
| (0.5 ppm) | Short; | (5 g/L) | Clean 100% | | | |
| | Temperature: | | cotton T-shirt | | | |
| | 30° C.; | | ballast. | | | |
| | Centrifuge: | | | | | |
| | 1600 rpm | | | | | |
| CBM72 | Cottons wash; | Liquid Ariel Color | T-shirts | Line | 1.61 +/− 0.18 | 1.87 +/− 0.02 |
| SEQ ID NO: 10 | Additional program: | and Style | Soiled SBL-CFT | dry | | |
| (0.5 ppm) | Short; | (5 g/L) | Clean 100% | | | |
| | Temperature: | | cotton T-shirt | | | |
| | 30° C.; | | ballast. | | | |
| | Centrifuge: | | | | | |
| | 1600 rpm | | | | | |

Values specify the average SA value rank given by the panel according to the AATCC smooth-ness standards +/− StE.

### Example 7

### CBM79—Anti-Crease Properties Evaluated on CS-10 Swatches

Eight pieces CS-10 swatches (CFT) in size 5×5 cm² were washed in Terg-o-Tometer 1 L beakers with 20 min wash and 10 min rinse under running tap water. Ariel Color & Style was dosed 5 g/L. A purified carbon-binding module from the CBM79 class was tested (SEQ ID NO: 8) dosed 0.5 ppm. 2 independent replica of each beaker were conducted. From each beaker CS-10 swatches were horizontally dried on filterpaper for 16 h at room temperature. Fabric pieces were evaluated by preference test of randomized pairs by a panel consisting of 14 untrained panelists. Panelists were asked to prefer the least wrinkled swatches. After evaluation, average across the panel scores was calculated for each condition.

| | Terg-o- | | Soiled/clean | | | |
|---|---|---|---|---|---|---|
| | Tometer wash | | Textile in | Drying | Preference test % preferred | |
| Protein | conditions | Detergent | the wash | regime | −CBM | +CBM |
| CBM79 | Temperature: | Model B | 8 CS-10 | Hand-squeezed | 18 | 82 |
| SEQ ID NO: 8 | 20° C.; | (3.3 g/L) | (CFT), | and horisontal | | |
| (0.5 ppm) | 25°dH | | 100% cotton ballast | drying on filter | | |
| | water hardness | | (mixed CS-11 and | paper at RT for 16 h | | |
| | and tap water | | WFK80A) to 30 g | | | |
| | for rinse | | | | | |

1-28. (canceled)

29. A method for reducing wrinkles and/or providing increased anti-crease properties and/or providing improved ease of ironing and/or providing improved shape retention in a cleaning process of a fabric or textile, comprising contacting the fabric or textile with a liquid solution comprising a polypeptide having carbohydrate binding activity.

30. The method of claim 29, wherein the liquid solution is a wash liquor.

31. The method of claim 29, provided as a laundry booster.

32. The method of claim 29, wherein the polypeptide is of microbial origin.

33. The method of claim 29, wherein the polypeptide having carbohydrate binding activity is selected from the group consisting of carbohydrate binding modules (CBM) and mixtures thereof.

34. The method of claim 29, wherein the carbohydrate binding module is derived from a polypeptide having glycoside hydrolase, xylanase, endoglucanase, activity.

35. The method of claim 29, wherein the carbohydrate binding module is selected from the group consisting of CBM family 1, 4, 17, 28, 30, 44, 72 and 79, and mixtures thereof.

36. The method of claim 33, wherein the CBM is selected from the group consisting of polypeptides having at least 60% sequence identity to one of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18.

37. The method of claim 29, where the wrinkles are reduced with at least 0.15 units when the textile is evaluated by the AATCC Smoothness standard Average SA-value according to AATCC.

38. The method of claim 29, wherein the anti-crease effect ratio of test panelists preferring fabrics washed with CBM vs test panelists preferring fabrics washed without CBM is at least 60:40.

39. The method of claim 29, wherein the improved softness effect ratio of test panelists preferring fabrics washed with CBM vs test panelists preferring fabrics washed without CBM is at least 60:40.

40. The method of claim 29, wherein the fabrics or textiles are cotton containing textiles.

41. A detergent composition, comprising a polypeptide having carbohydrate binding activity which is a carbohydrate binding module (CBM).

42. The detergent composition of claim 41, wherein the carbohydrate binding modules are selected from the group consisting of CBM family 1, 4, 17, 28, 30, 44, 72 or 79.

43. The detergent composition of claim 41, wherein the CBM is selected from the group consisting of polypeptides having at least 60% sequence identity to one of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18.

44. The detergent composition of claim 41, wherein the CBM is attached to another polypeptide.

45. The detergent composition of claim 41, wherein the CBM is not attached to an enzyme.

46. The detergent composition of claim 41, further comprising one or more enzymes selected among protease, lipase, cutinase, amylase, carbohydrase, cellulase, pectinase, mannanase, arabinase, galactanase, xylanase, oxidase, nuclease, laccase, and/or peroxidase.

47. The detergent composition of claim 42, further comprising one or more cleaning composition components, selected from surfactants, builders, co-builders, polymers, bleaching agents, fabric huing agents and/or perfumes.
